(19) United States
(12) Patent Application Publication
(10) Pub. No.: US 2013/0048136 A1
(43) Pub. Date: Feb. 28, 2013

(54) COPOLYAMIDE COMPOSITIONS DERIVED FROM TRIACYLGLYCERIDES

(71) Applicants: EKATERINI KOROVESSI, Wilmington, DE (US); Anna Kutty Mathew, Kingston (CA); Marvin M. Martens, Bettendorf, IA (US); Joachim C. Ritter, Wilmington, DE (US)

(72) Inventors: EKATERINI KOROVESSI, Wilmington, DE (US); Anna Kutty Mathew, Kingston (CA); Marvin M. Martens, Bettendorf, IA (US); Joachim C. Ritter, Wilmington, DE (US)

(73) Assignee: E I DU PONT DE NEMOURS AND COMPANY, Wilmington, DE (US)

(21) Appl. No.: 13/596,103

(22) Filed: Aug. 28, 2012

Related U.S. Application Data
(60) Provisional application No. 61/528,425, filed on Aug. 29, 2011.

(51) Int. Cl. C08L 77/06; C08G 69/26; F16L 11/06; C12P 13/02

(52) U.S. Cl. 138/118; 435/129; 528/363; 524/607

(57) ABSTRACT

Disclosed is a copolyamide including at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; wherein at least one repeat unit is selected from structures (I) and (II); and any one the repeat units may be present up to about 92 mole percent. Also disclosed is a process for providing the copolyamide; and thermoplastic compositions and tubing including the copolyamides.

### CROSS REFERENCE TO RELATED APPLICATIONS

This application claims priority of Application No. 61/528,425, filed Aug. 29, 2011.

### FIELD OF THE INVENTION

The present invention relates to the field of polyamide compositions derived from triacylglycerides, such as vegetable and/or animal fats and/or oils.

### BACKGROUND OF INVENTION

Polymeric materials, including thermoplastics and thermosets, are used extensively as automotive components and as molded articles for various other applications. They are light weight and relatively easy to mold into complex parts, and are therefore preferred over metals in many such applications. However, a problem encountered by some polymers is salt stress (induced) corrosion cracking (SSCC), where a polymeric part in stress undergoes accelerated corrosion when exposed to inorganic salts. This often results in cracking and premature failure of the molded parts. Polymeric molded parts may also need to exhibit significant high durability and toughness under use conditions.

Polyamides, such as polyamide 6,6, polyamide 6, polyamide 6,10 and polyamide 6,12 have been made into and used as vehicular interior and exterior components and in the form of other parts. While it has been reported that polyamides 6,10 and 6,12 are satisfactorily resistant to SSCC (see for instance Japanese Patent 3271325B2), all of these polyamides are prone to SSCC in such uses, because for instance, various sections of vehicles and their components are sometimes exposed to salts, for example sodium chloride or calcium chloride, used to melt snow and ice in colder conditions. Corrosion of metallic parts such as fittings and frame components made from steel and various iron based alloys in contact with water and road salts can also lead to formation of salts. These salts, in turn, can further attack the polyamide based automotive parts, making them susceptible to SSCC. Thus polyamide compositions with improved resistance to SSCC are desired.

U.S. Pat. No. 4,076,664 discloses a terpolyamide resin that has favorable resistance to zinc chloride.

US 2005/0234180 discloses a resin molded article having an excellent snow melting salt resistance, said article comprising 1 to 60% by weight of aromatic polyamide resin.

Furthermore, increasing fossil raw material prices and to reduce greenhouse gas emissions to environment make it desirable to develop engineering polymers from linear, long chain dicarboxylic acids prepared from renewable feedstocks. As such, there is a demand for renewable bio-based polymers having similar or better performance characteristics than petrochemical-based polymers. As example, renewable nylon materials such as PA 610 are based on ricinoleic acid derived sebacic acid (C10). However, ricinoleic acid production requires the processing of castor beans and involves the handling of highly allergenic materials and highly toxic ricin. Moreover, the production of sebacic acid is further burdened with high consumption of energy and with the formation of a large amount of salts as by products associated with other byproducts.

WO 2010/068904 discloses a method to produce renewable alkanes from biomass based triacylglycerides in high yield and selectivity and their subsequent fermentation to renewable diacids. Such naturally occurring triacylglycerides, also referred to as oils and fats, are composed of a glycerol backbone esterified with three fatty acids of a variety of chain lengths specific to the type of fats and oils. Most abundant amongst vegetable oils are triacylglycerides based on C12, 14, 16 and C18 fatty acids. Several vegetable oils are rich in C12, C14, C16 and C18 fatty esters including soybean oil, palm oil, palm kernel oil, sunflower oil, olive oil, cotton seed oil, rape seed oil, and corn oil (Ullmann's Encyclopedia of Technical Chemistry, A. Thomas: “Fats and Fatty Oils” (2005 Wiley-VCH Verlag GmbH & Co. KGaA, Weinheim, electronic version, 10.1002114356007.a10 173). As such, dioic acid streams based on the oxidative fermentation of renewable alkanes derived from such oils, being rich in C16 and C18 dioic acids, may be useful in formation of economically attractive polymers.

The vegetable oils contain usually at most 50% C12 components (Ullman ref.), hence other diacid components are usually separated out from the mixture of acids and used for other purposes. In order to improve the economics of renewable diacids processes based on triacylglyceride hydrogenation and fermentation of the resulting n-alkanes, it is desirable to include all long chain diacid components without isolation into polyamide chains. Hence, developing sustainable compositions of renewable polyamide copolymers, containing mixtures of long chain diacid which avoid the isolation steps of diacids meet or exceed the performance requirement of existing commercial long chain polyamide compositions at competitive cost is a highly desirable goal.

### SUMMARY OF THE INVENTION

Disclosed is a copolyamide comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; wherein at least one repeat unit is selected from structures (I) and (II); and any one the repeat units may be present up to about 92 mole percent.

Another embodiment is a copolyamide consisting essentially of at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);
and n is an integer selected from 4, 6, 10 or 12; wherein at least one repeat unit is selected from structures (I) and (II); and any one the repeat units may be present up to about 92 mole percent.

Another embodiment is a process for making a copolyamide comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; and any one the repeat units may be present up to about 92 mole percent, comprising the steps of;
a) fermenting a mixture of at least two long chain linear alkanes to a mixture of at least two long chain linear dicarboxylic acids of C12, C14, C16 and C18 carbon atoms; and
b) polymerizing the mixture of at least two long chain dicarboxylic acids containing C12, C14, C16 and C18 carbon atoms and a diamine to provide said copolyamide.

Another embodiment is a thermoplastic composition comprising:

(a) a copolyamide comprising at least two repeat units selected from the group consisting of structures (I) to (IV) as disclosed above:
(b) 0 to 30 weight percent of at least one polymeric toughener; and
(c) 0 to 10 weight percent of functional additives; and
(d) 0 to 60 weight percent of at least one reinforcing agent;
wherein the weight percent of (a), (b), (c) and (d) are based on the total weight of the thermoplastic composition and at least one component of the group consisting of (b), (c), (d), or a combination of these, is present in at least 0.1 weight percent.

Another embodiment is a flexible tubing comprising:

(a) a copolyamide comprising at least two repeat units as disclosed above;
(b) 0 to 30 weight percent of at least one polymeric toughener;
(d) 0 to 10 weight percent thermal stabilizer; and
(e) 0 to 20 weight percent of plasticizer; and
wherein the weight percent of (a), (b), and (d) and (e) are based on the total weight of the thermoplastic composition.

### BRIEF DESCRIPTION OF FIGURES

FIG. 1 shows a dynamic mechanical analysis of a crystalline copolymer.

### DETAILED DESCRIPTION OF THE INVENTION

Herein melting points are as determined using differential scanning calorimetry (DSC) at a scan rate of 10° C./min in the first heating scan, wherein the melting point is taken at the maximum of the endothermic peak, and the heat of fusion in Joules/gram (Jig) is the area within the endothermic peak.

Herein freezing points are as determined with DSC in the cooling cycle at a scan rate of 10° C./min carried out after the first heating cycle as per ASTM D3418.

Herein the term delta melting point minus freezing point (MP-FP, in ° C.) is the difference between the melting point and freezing point of a particular polymer or copolymer, wherein the melting point and freezing point are determined as disclosed above. The term delta MP-FP is one measure of the crystallinity of polymer or copolymer and, in part, determines the crystallization kinetics of the polymer or copolymer. A low delta MP-FP typically gives high crystallization rates; and faster cycle times in injection molding process. A low delta MP-FP typically gives desirable high temperature properties in extrusion processing as well.

Dynamic mechanical analysis (DMA) is used herein for determination of storage modulus (E′) and loss modulus (E″), and glass transition, as a function of temperature. Tan delta is a curve resulting from the loss modulus divided by the storage modulus (E″/E′) as a function of temperature.

Dynamic mechanical analysis is discussed in detail in “Dynamic Mechanical Analysis: A practical Introduction,” Menard K. P., CRC Press (2008) ISBN is 978-1-4200-5312-8. Storage modulus (E′), loss modulus (E″) curves exhibit specific changes in response to molecular transitions occurring in the polymeric material in response to increasing temperature. A key transition is called glass transition. It characterizes a temperature range over which the amorphous phase of the polymer transitions from glassy to rubbery state, and exhibits large scale molecular motion. Glass transition temperature is thus a specific attribute of a polymeric material and its morphological structure. For the co-polyamide compositions disclosed herein, the glass transition occurs over a temperature range of about 20 to about 50° C. The Tan delta curve exhibits a prominent peak in this temperature range. This peak tan delta temperature is defined in the art as the tan delta glass transition temperature, and the height of the peak is a measure of the crystallinity of the polymeric material. A polymeric sample with low or no crystallinity exhibits a tall tan delta peak due to large contribution of the amorphous phase molecular motion, while a sample with high level of crystallinity exhibits a smaller peak because molecules in crystalline phase are not able to exhibit such large scale rubbery motion. Thus, herein the value of tan delta glass transition peak is used as a comparative indicator of level of crystallinity in the co-polyamides and melt-blended thermoplastic polyamide compositions.

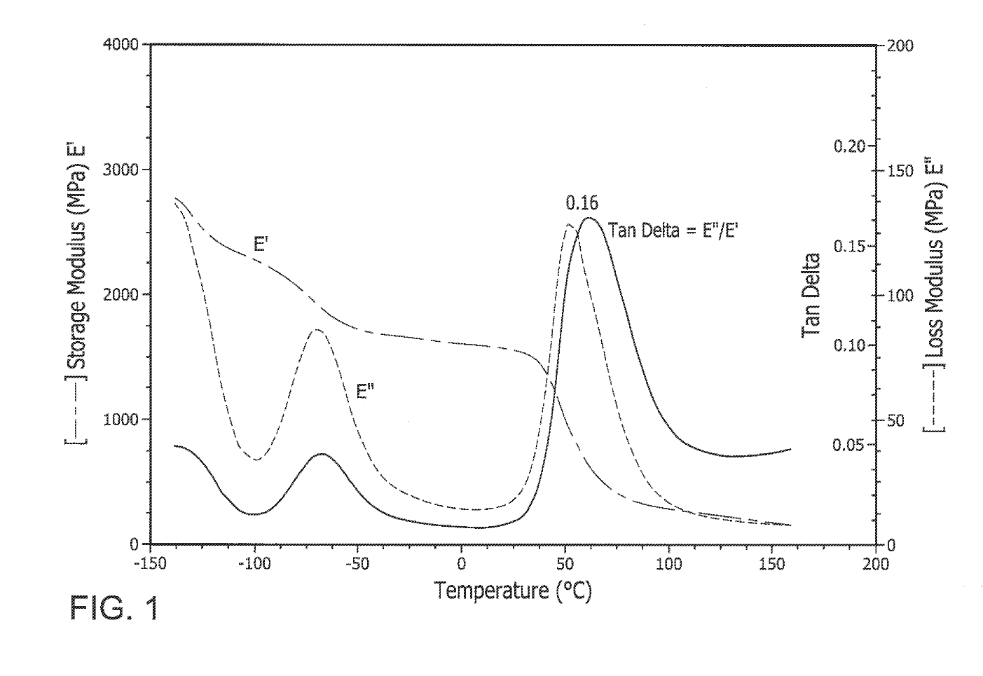

FIG. 1 shows a dynamic mechanical analysis of a crystalline co-polymer showing the storage modulus (E′), loss modulus (E″) curves and computed tan delta curve (E″/E′). A higher tan delta peak corresponds to lower crystallinity and conversely, a lower tan delta peak corresponds to higher crystallinity; as discussed in “Thermal Analysis of Polymers,” Sepe M. P., Rapra Review Reports, Vol. 8, No. 11 (1977).

The co-polyamides disclosed herein have two or more amide repeat units in polymer chains. The co-polyamides are identified by their respective repeat units. The following list exemplifies the abbreviations used to identify monomers and repeat units in polyamides and co-polyamides (PA) disclosed herein:
DMD Decamethylenediamine
HMD Hexamethylene diamine (or 6 when used in combination with a diacid)
TMD 1,4-Tetramethylene diamine (or 4 when used in combination with a diacid)
6 [symbol] -Caprolactam
AA Adipic acid
Decanedioic acid (DDA)
12 Dodecanedioic acid (DDDA)
14 Tetradecanedioic acid
16 Hexadecanedioic acid
18 Octadecanedioic acid
66 Polymer repeat unit formed from HMD and AA
610 Polymer repeat unit formed from HMD and DDA
612 Polymer repeat unit formed from HMD and DDDA
614 Polymer repeat unit formed from HMD and tetradecanedioic acid
616 Polymer repeat unit formed from HMD and hexadecanedioic acid
618 Polymer repeat unit formed from HMD and octadecanedioic acid
6 Polymer repeat unit formed from ε-caprolactam
11 Polymer repeat unit formed from 11-aminoundecanoic acid
12 Polymer repeat unit formed from 12-aminododecanoic acid

Note that in the art, the term 6 when used alone designates a polymer repeat unit formed from ε-caprolactam, contains acid and amine in the same monomer unit. In polyamide repeat units comprising a diamine and a diacid, the number of carbon atoms in the diamine is designated first, followed by the number of carbon atoms in the diacid; for instance, in 66, the first 6 refers to the number of carbon atoms in the diamine, HMD, and the second 6 refers to number of carbon atoms in diacid, adipic acid. Likewise, repeat units derived from other amino acids or lactams are designated as single numbers designating the number of carbon atoms.

In co-polyamides, the repeat units are separated by a slash (that is, /). For instance poly(hexamethylene decanediamide/decamethylene decanediamide) is abbreviated by PA610/1010 (75/25), and the values in brackets are the mole % of each repeat unit in the co-polyamide.

In various embodiments of the invention, the co-polyamides disclosed herein, comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; wherein at least one repeat unit is selected from structures (I) and (II); and any one the repeat units may be present up to about 92 mole percent.

In one embodiment, the co-polyamides have 8 to 92 mole percent of repeat units of structure (I), and 8 to 92 mole percent of repeat units of structure (II).

In another embodiment, the co-polyamides have 60 to 92 mole percent of repeat units of structure (I), and 8 to 40 mole percent of repeat units of structure (II).

In one embodiment, the co-polyamides have, 8 to 92 mole percent of repeat units of structure (II), and 8 to 92 mole percent of repeat units of structure (III).

In another embodiment in the invention, the co-polyamides have 20 to 80 mole percent of repeat units of structure (II), and 20 to 80 mole percent of repeat units of structure (III).

In one embodiment, the co-polyamides have 8 to 92 mole percent of repeat units of structure (I), and 8 to 92 mole percent of repeat units of structure (III).

In another embodiment, the co-polyamides have 60 to 92 mole percent of repeat units of structure (I) and, 8 to 40 mole percent of repeat units of structure (III).

In one embodiment, the co-polyamides have 8 to 92 mole percent of repeat units of structure (I), and 8 to 92 mole percent of repeat units of structure (IV).

In another embodiment, the co-polyamides have 60 to 92 mole percent repeat units of structure (I), and 8 to 40 mole percent of repeat units of structure (IV).

In one embodiment, the co-polyamides have 8 to 92 mole percent of repeat units of structure (I), and 8 to 92 mole percent of repeat units of structure (IV).

In another embodiment, the co-polyamides have 60 to 92 mole percent repeat units of structure (II), and 8 to 40 mole percent of repeat units of structure (IV).

In one embodiment, the co-polyamides have 4 to 92 mole percent of repeat units of structure (I), 4 to 92 mole percent of repeat units of structure (II), and 4 to 92 mole percent of repeat units of structure (II).

In one embodiment, the co-polyamides have 40 to 80 mole percent of repeat units of structure (I), 15 to 30 mole percent of repeat units of structure (II), and 5 to 30 mole percent of repeat units of structure (III).

In one embodiment, the co-polyamides have 4 to 92 mole percent of repeat units of structure (I), 4 to 92 mole percent of repeat units of structure (II), and 4 to 92 mole percent of repeat units of structure (IV).

In one embodiment, the co-polyamides have 40 to 80 mole percent of repeat units of structure (I), 15 to 30 mole percent of repeat units of structure (II), and 5 to 30 mole percent of repeat units of structure (IV).

In one embodiment, the co-polyamides have 4 to 92 mole percent of repeat units of structure (I), 4 to 92 mole percent of repeat units of structure (III), and 4 to 92 mole percent of repeat units of structure (IV).

In one embodiment, the co-polyamides have 40 to 80 mole percent of repeat units of structure (I), 15 to 30 mole percent of repeat units of structure (III), and 5 to 30 mole percent of repeat units of structure (IV).

In one embodiment, the co-polyamides have 4 to 92 mole percent of repeat units of structure (II), 4 to 92 mole percent of repeat units of structure (III), and 4 to 92 mole percent of repeat units of structure (IV).

In one embodiment, the co-polyamides have 40 to 80 mole percent of repeat units of structure (II), 15 to 30 mole percent of repeat units of structure (III), and 5 to 30 mole percent of repeat units of structure (IV).

In one embodiment, the co-polyamides have 2 to 92 mole percent of repeat units of structure (I), 2 to 92 mole percent of repeat units of structure (II), 2 to 92 mole percent of repeat units of structure (III), and 2 to 92 mole percent of repeat units of structure (IV).

In one embodiment, the co-polyamides have 40 to 75 mole percent of repeat units of structure (I), 10 to 30 mole percent of repeat units of structure (II) 5 to 25 mole percent of repeat units of structure (III), and 5 to 10 mole percent of repeat units of structure (IV).

In all the above embodiments the diamine may be selected from the group consisting of 1,4-tetramethylene diamine (TMD), 1,6-hexamethylene diamine (HMD); 1,10-decamethylene diamine (DMD) and, 1,12-dodecamethylene diamine (DDMD). Preferred embodiments are any of those copolyamides disclosed above wherein n is 6.

The term “comprising” means that the embodiment encompasses all the elements listed, but may also include additional unnamed, unrecited elements. Herein, for instance, the term as applied to the co-polyamide, means the co-polyamide includes diacids containing C12, C14, C16 and C18 carbon atom and diamine containing 4, 6, 10 and 12 carbon atoms, but may also include other additional, unnamed, unrecited components.

The term “consisting of” means the embodiment necessarily includes the listed components only and no other unlisted components are present. Herein, for instance, the term as applied to the co-polyamide, means the co-polyamide includes the repeat units of formula (I) to (IV), and does not include any other repeat units.

The term “consisting essentially of” means the embodiment necessarily includes the listed components only but may also include additional unnamed, unrecited elements, which does not materially affect the basic and novel characteristic of the composition. Herein, for instance, the term as applied to the copolyamide, means the copolyamide includes the repeat units of structure (I) and (II), or (I) and (III) or (I) and (IV) or (II) and (II) or (II) and (IV) or (I) and (II) and (II) or (I) and (III) and (IV) or (II) and (III) and (IV) or (I) and (II) and (III) and (IV) but may include any other repeat units, which do not affect the novel characteristics of the composition.

The copolyamides of the invention are preferably prepared from aliphatic dioic acids and aliphatic diamines, at least one of which is bio-sourced or “renewable”. By “bio-sourced” is meant that the primary feed-stock for preparing the dioic acid and/or diamine is a renewable biological source, for instance, vegetable matter including grains, vegetable oils, cellulose, lignin, fatty acids; and animal matter including fats, tallow, oils such as whale oil, fish oils, and the like. These bio-sources of dioic acids and aliphatic diamines have a unique characteristic in that they all possess high levels of the carbon isotope ¹⁴C (carbon pools having an elevated content of ¹⁴C are sometimes referred to as “modern carbon”); as compared to fossil or petroleum sources of the dioic acids and aliphatic diamines. This unique isotope feature remains unaffected by non-nuclear, conventional chemical modifications. Thus the ¹⁴C isotope level in bio-sourced materials provides an unalterable feature that allows any downstream products, such as polyamides; or products comprising the polyamides, to be unambiguously identified as comprising a bio-sourced material. Furthermore, the analysis of ¹⁴C isotope level in dioic acids, diamines and downstream product is sufficiently accurate to verify the percentage of bio-sourced carbon in the downstream product.

The copolyamides of various embodiments preferably has a carbon content wherein the carbon content comprises at least 50 percent modern carbon (pMC), as determined with the ASTM-D6866 Biobased determination method. In other embodiments the polyamide has a modern carbon content of at least 60, 65, 70, 75, 80, and 85 pMC, respectively, as determined with the ASTM-D6866 Method.

The ASTM-D6866 method to derive a “Biobased content” is built on the same concepts as radiocarbon dating, but without use of the age equations. The method relies on determining a ratio of the amount of radiocarbon (¹⁴C) in an unknown sample to that of a modern reference standard. The ratio is reported as a percentage with the units “pMC” (percent modern carbon). If the material being analyzed is a mixture of present day radiocarbon and fossil carbon (fossil carbon being derived from petroleum, coal, or a natural gas source), then the pMC value obtained correlates directly to the amount of biomass material present in the sample.

The modern reference standard used in radiocarbon dating is a National Institute of Standards and Technology—USA (NIST-USA) standard with a known radiocarbon content equivalent approximately to the year AD 1950. AD 1950 was chosen since it represented a time prior to thermo-nuclear weapons testing which introduced large amounts of excess radiocarbon into the atmosphere with each explosion (termed “bomb carbon”). This was a logical point in time to use as a reference for archaeologists and geologists. For those using radiocarbon dates, AD 1950 equals “zero years old”. It also represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. It's gradually decreased over time with today's value being near 107.5 pMC. This means that a fresh biomass material such as corn, vegetable oils, etc, and materials derived therefrom, would have a radiocarbon signature near 107.5 pMc.

The radiocarbon dating isotope (¹⁴C), with its nuclear half life of 5730 years, clearly allows one to apportion specimen carbon between fossil carbon (“dead”) and biospheric (“alive”) feedstocks. Fossil carbon, depending upon its source, has very close to zero ¹⁴C content.

Combining fossil carbon with present day carbon into a material will result in a dilution of the present day pMC content. By presuming 107.5 pMC represents present day biomass materials and 0 pMC represents petroleum (fossil carbon) derivatives, the measured pMC value for that material will reflect the proportions of the two component types. Thus, a material derived 100% from present day vegetable oil would give a radiocarbon signature near 107.5 pMC. If that material was diluted with 50% petroleum derivatives, it would give a radiocarbon signature near 54 pMC.

The results provided by the ASTM D6866 method are the Mean Biobased Result and encompasses an absolute range of 6% (plus and minus 3% on either side of the Mean Biobased Result) to account for variations in end-component radiocarbon signatures. It is presumed that all materials are present day or fossil in origin. The result is the amount of biobased component “present” in the material, not the amount of biobased material “used” in the manufacturing process.

A biomass content result is derived by assigning 100% equal to 107.5 pMC and 0% equal to 0 pMC. In this regard, a sample measuring 99 pMC will give an equivalent Biobased content result of 93%. This value is referred to as the “Mean Biobased Result” and assumes all the components within the analyzed material were either present day living or fossil in origin

Several commercial analytical laboratories have capabilities to perform ASTM-D6866 method. The analyses herein were conducted by Beta Analytics Inc. Miami Fla., USA.

Another embodiment of the invention is a process of making copolyamides comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; and any one the repeat units may be present up to about 92 mole percent, comprising the steps of:
a) fermenting a mixture of at least two long chain linear alkanes to a mixture of at least two long chain linear dicarboxylic acids of C12, C14, C16 and C18 carbon atoms;
b) polymerizing the mixture of at least two long chain dicarboxylic acids containing C12, C14, C16 and C18 carbon atoms and a diamine to yield the co-polyamide.

The mixture of at least two long chain linear alkanes are selected from the group consisting of C12, C14, C16 and C18 linear alkanes; and the mixture comprises up to 92 mol % of a first linear alkane and at least 8 mol % of a second linear alkane. In one embodiment the mixture of linear alkanes comprises 60 to 92 mol % of a first linear alkane and 8 to 40 mol % of a second linear alkane.

Preferably the mixture of at least two long chain linear alkanes is derived from a renewable bio-source. A renewable bio-source of mixtures of linear alkanes, useful in the process disclosed above, is the catalytic hydrotreating of fatty acids and fatty esters including triglycerides. A suitable hydrotreating process is disclosed in WO 2010/068904 and provides mixtures of C12, C14, C16 and C18 linear alkanes in high yield, depending upon the fatty acid; fatty ester; and/or triglyceride feedstock. The renewable mixture of long chain linear alkanes; and renewable linear dicarboxylic acids derived therefrom by fermentation, are derived fatty acids or fatty esters including triacylglycerides. Preferably the renewable mixture of long chain linear alkanes; and the renewable linear dicarboxylic acids derived therefrom by fermentation, are derived from vegetable oils selected from the group consisting of palm oil, palm kernel oil, rapeseed oil, soybean oil, cottonseed oil, peanut oil, olive oil, coconut oil, castor oil, canola oil, and sunflower oil; animal fats selected from the group consisting of poultry fats, pork fat, horse fat, yellow grease, and tallow; any combination of vegetable oils; any combinations of animal fats; and any combination of vegetable oils and animal fats.

Fermenting a mixture of long chain linear alkanes provides a mixture of at least two long chain dicarboxylic acids of C12, C14, C16 and C18 carbon atoms. The mixture of at least two aliphatic dicarboxylic acids can be isolated from the fermentation broth using well known procedures described in the art. For instance, GB patent 1,096,326, disclose the ethyl acetate extraction of a fermentation broth, followed by esterification of the extract with methanol and sulfuric acid catalysis to provide the corresponding dimethyl ester of the dicarboxylic acid.

A mixture of linear dicarboxylic acids derived from a fermentation process mixture maybe used in forming a copolyamide without separating individual linear dicarboxylic acids.

Preferably in the process disclosed above, the linear alkanes, produced from fatty acids or fatty esters including triacylglycerides, are not separated, but are subjected to fermentation as a mixture of alkanes, providing a mixture of dicarboxylic acids. The Cₙ composition of the mixture of dicarboxylic acids corresponds substantially to the Cₙ molar composition of the fatty acid or fatty ester chosen as feedstock. The mixture of dicarboxylic acids may be used in polymerization with a diamine of choice H₂N(CH₂)ₙNH₂, wherein n is selected from 4, 6, 10 or 12, to yield a co-polyamide comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II); and

—C(O)(CH₂)₁₆C(O)NH(CH₂)ₙNH— (IV);

and n is an integer selected from 4, 6 10 or 12; and any one the repeat units may be present up to about 92 mole percent.

Table I shows the fatty acid chain lengths of the triacylglyceride and fatty acid sources (by weight percent) for the feeds used in most of the example. The fatty acid chain lengths in Table I, are given in lipid nomenclature of the form C:D, where C is the number of carbon atoms in the fatty acid and D is the number of double bonds in the fatty acids. For example,

C_{18:1} refers to an 18 carbon chain with 1 unsaturated bond, C_{18:2} refers to an 18 carbon chain with 2 unsaturated bond, and C_{18:3} refers to an 18 carbon chain with 3 unsaturated bond. In Table I,
C₁₈₊ refers to fatty acids containing greater than 18 carbons. The values in Table I, are representative of the triacylglyceride content of indicated oils, which can vary from sample to sample.

TABLE I
Fatty acid chain lengths of triacylglycerides and fatty acid sources by weight percent

| | | Rapeseed | Rapeseed | | Palm | | | | Cotton | | |
|---|---|---|---|---|---|---|---|---|---|---|---|
| | Soybean | oil, Low | oil (high | Palm | kernel | Sunflower | Olive | Coconut | seed | Peanut | Corn |
| | Oil | Erucic | erucic) | oil | oil | Oil | oil | oil | oil | oil | oil |
| Fatty acid | ~96 | ~100 | ~100 | ~100 | ~100 | ~100 | ~100 | ~100 | ~100 | ~100 | ~95 |
| trigycerides | | | | | | | | | | | |
| (%) | | | | | | | | | | | |
| C8:0, | | | | | 3 | | | 7 | | | |
| Caprylic | | | | | | | | | | | |
| C10:0, Capric | | | | | 3 | | | 6 | | | |
| C12:0, Lauric | | | | | 50 | | | 50 | | | |
| C14:0, | | | | 1 | 18 | | | 18 | 1 | | |
| Myristic | | | | | | | | | | | |
| C16:0, | 11 | 5 | 4 | 45 | 8 | 8 | 12 | 9 | 24 | 12 | 8-12 |
| Palmitic | | | | | | | | | | | (12) |
| C16:1, | | | | | | | 2 | | | | |
| Palmitoleic | | | | | | | | | | | |
| C18:0, | 2 | 2 | 2 | 4 | 2 | 2 | 2 | 3 | 3 | 3 | 2-5 |
| Stearic | | | | | | | | | | | (2.5) |
| C18:1, | 22 | 63 | 19 | 40 | 14 | 20 | 70 | | 17 | 52 | 19-49 |
| Oleic | | | | | | | | | | | (29) |
| C18:2, | 55 | 20 | 14 | 10 | 2 | 68 | 13 | 1 | 55 | 27 | 34-62 |
| Linoleic | | | | | | | | | | | (56) |
| C18:3, | 6 | 9 | 8 | | | | 1 | 1 | | | |
| Linolenic | | | | | | | | | | | |
| Total C₁₈ | 85 | 94 | 43 | 54 | 18 | 90 | 86 | 5 | 75 | 82 | — |
| (%) | | | | | | | | | | | |
| C20:0, | | | | | | | 1 | | | 2 | |
| Arachidic | | | | | | | | | | | |
| C20:1, | 1 | | 13 | | | | | | | 2 | |
| Eicosenoic | | | | | | | | | | | |
| Total C₂₀ | 1 | | 13 | | | | 1 | | | 4 | |
| (%) | | | | | | | | | | | |
| C22:0, | | | | | | | | | | 3 | |
| Behenic | | | | | | | | | | | |
| C22:1, | | | 40 | | | | | | | | |
| Erudic | | | | | | | | | | | |
| Total C₂₂ | | | 40 | | | | | | | 3 | |
| (%) | | | | | | | | | | | |
| C24:0, | | | | | | | | | | 1 | |
| Lignoceric | | | | | | | | | | | |

The above oils and fats may be used for hydrotreating and fermentation to form corresponding dicarboxylic acids.

Methods and microorganisms for fermenting linear alkanes to linear dicarboxylic acids are known, such as those described, for example, in U.S. Pat. Nos. 5,254,466; 5,620,878; 5,648,247, 7,405,063 and Published Application US 2004/0146999 (each of which is by this reference incorporated in its entirety as a part hereof for all purpose); and in EP 1 273 663.

Fermentation of a mixture of at least two long chain linear alkanes to a mixture of at least two long chain linear dicarboxylic acids of C12, C14, C16 and C18 carbon atoms may be by any suitable biocatalyst having alkane hydroxylating activity.

Particularly suitable as biocatalysts are microorganisms that are genetically engineered for enhanced alkane hydroxylating activity. The enhanced hydroxylating activity may be due to enhanced alkane monooxygenase, fatty acid monooxygenase or cytochrome P450 reductase separately or in various combinations. For example, suitable biocatalysts may be microorganisms such as yeast of the genera Candida, Pichia, or Saccharomyces that have been genetically engineered to express increased cytochrome P450 monooxygenase activity and/or increased cytochrome P450 reductase activity. Separately or in addition, a suitable biocatalyst may be genetically engineered to disrupt the β-oxidation pathway. Disrupting the β-oxidation pathway increases metabolic flux to the ω-oxidation pathway and thereby increases the yield and selectivity of a bioprocess for conversion of alkanes to mono- and diterminal carboxylates. As an example, US Published Application 2004/0146999 discloses a process for the bioproduction of C₆ to C₂₂ mono- and di-carboxylic acids by contacting, under aerobic conditions, transformed Pichia pastoris characterized by a genetically engineered enhanced alkane hydroxylating activity or transformed Candida maltosa characterized by a genetically engineered enhanced alkane hydroxylating activity with at least one C₆ to C₂₂ straight chain hydrocarbon in the form CH₃ (CH₂)ₓCH₃ wherein x=4 to 20. The reference also discloses a transformed Pichia pastoris comprising at least one foreign gene encoding a cytochrome P450 monooxygenase and at least one foreign gene encoding a cytochrome P450 reductase, each gene operably linked to suitable regulatory elements such that alkane hydroxylating activity is enhanced. Also disclosed are genetically-engineered Candida maltosa strains that have enhanced cytochrome P450 activity and/or gene disruptions in the β-oxidation pathway. Genetic engineering may be as described in US Published Application 2004/0146999 or by additional methods well known to one skilled in the art. Known promoters, coding regions, and termination signals may be used for expression of enzyme activities. The fermentation of a mixture of at least two long chain linear alkanes to a mixture of at least two long chain linear dicarboxylic acids containing C12, C14, C16 and C18 carbon atoms, wherein the long chain dicarboxylic mixture at least contain either C12 or C14 carbon atoms may be carried out using transformed pichia pastoris or transformed candia maltosa strain.

In another embodiment, a mixture of biocatalyst each having a selectivity towards an individual alkane are used.

Preferred copolyamides comprises at least two repeat units selected from the group consisting of structures (I) to (IV) are prepared from C12, C14, C16 and C18 dioic acids derived from fermentation of mixture of linear alkanes, wherein the mixture of linear alkanes are derived from vegetable oils selected from the group consisting of palm oil, palm kernel oil, rapeseed oil, soybean oil, cottonseed oil, peanut oil, olive oil, coconut oil, castor oil, canola oil, and sunflower oil; animal fats selected from the group consisting of poultry fats, pork fat, horse fat, yellow grease, and tallow; any combination of vegetable oils; any combinations of animal fats; and any combination of vegetable oils and animal fats.

Another embodiment of the invention is a thermoplastic composition comprising:
a) a copolyamide, preferably at least 30 to 80 wt %, comprising at least two repeat units from structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II); and

—C(O)(CH₂)₁₆C(O)NH(CH₂)ₙNH— (IV);

and n is an integer selected from 4, 6, 10 or 12; wherein at least one repeat unit is selected from structures (I) and (II); and any one the repeat units may be present up to about 92 mole percent;
b) 0 to 60 wt % of at least one reinforcing agent;
c) 0 to 30 wt % of at least one polymeric toughener;
d) 0 to 10 wt % of a functional additive;
wherein, the weight percent of a), b), c), and d) are based on the total weight of the thermoplastic composition and at least one component of the group consisting of (b), (c), (d), or a combination of these, is present in at least 0.1 weight percent.

Another embodiment of the invention is a thermoplastic composition consisting essentially of components (a), (b), (c) and (d), as disclosed above.

All the embodiments disclosed above for the co-polyamide comprising at least two repeat units from structures (I) to (IV) are also applicable to the thermoplastic composition.

The thermoplastic composition may optionally comprise 0.1 to about 60 weight percent, and preferably about 10 to 60 weight percent, 15 to 50 weight percent and 20 to 45 weight percent, of one or more reinforcement agents. The reinforcement agent may be any filler, but is preferably selected from the group consisting of calcium carbonate, glass fibers with circular cross-section, glass fibers with noncircular cross-section, glass flakes, glass beads, glass balloons, carbon fibers, talc, mica, wollastonite, calcined clay, kaolin, diatomite, magnesium sulfate, magnesium silicate, barium sulfate, titanium dioxide, boron nitrite, sodium aluminum carbonate, barium ferrite, potassium titanate and mixtures thereof. Glass fibers, glass flakes, talc, and mica are preferred reinforcement agents.

The thermoplastic composition may, optionally, comprise 0.1 to 30 weight percent of a polymeric toughener comprising a reactive functional group and/or a metal salt of a carboxylic acid. In one embodiment the thermoplastic composition comprises 2 to 20 weight percent, and preferably 6 to 15 weight %, polymeric toughener selected from the group consisting of: a copolymers of ethylene, glycidyl(meth)acrylate, and optionally one or more (meth)acrylate esters; an ethylene/α-olefin or ethylene/α-olefin/diene copolymer grafted with an unsaturated carboxylic anhydride; a copolymer of ethylene, 2-isocyanatoethyl(meth)acrylate, and optionally one or more (meth)acrylate esters; and a copolymer of ethylene and acrylic acid reacted with a Zn, Li, Mg or Mn compound to form the corresponding ionomer.

The thermoplastic composition may include 0 to 10 weight percent of functional additives such as thermal stabilizers, plasticizers, colorants, lubricants, mold release agents, and the like. Such additives can be added according to the desired properties of the resulting material, and the control of these amounts versus the desired properties is within the knowledge of the skilled artisan

The thermoplastic composition may include a thermal stabilizer selected from the group consisting of polyhydric alcohols having more than two hydroxyl groups and having a number average molecular weight (Mₙ) of less than 2000; one or more polyhydroxy polymer(s) having a number average molecular weight of at least 2000 and selected from the group consisting of ethylene/vinyl alcohol copolymer and poly(vinyl alcohol; organic stabilizer(s) selected from the group consisting of secondary aryl amines and hindered amine light stabilizers (HALS), hindered phenols and mixtures of these; copper salts; and mixtures these.

The thermoplastic composition may comprise 0.1 to 10 weight percent, and preferably 1 to 8 weight percent and 2 to 6 weight percent, of one or more polyhydric alcohols having more than two hydroxyl groups and having a number average molecular weight (Mₙ) of less than 2000 of less than 2000 as determined for polymeric materials with gel permeation chromatography (GPC).

Polyhydric alcohols may be selected from aliphatic hydroxylic compounds containing more than two hydroxyl groups, aliphatic-cycloaliphatic compounds containing more than two hydroxyl groups, cycloaliphatic compounds containing more than two hydroxyl groups, aromatic and saccharides.

Preferred polyhydric alcohols include those having a pair of hydroxyl groups which are attached to respective carbon atoms which are separated one from another by at least one atom. Especially preferred polyhydric alcohols are those in which a pair of hydroxyl groups is attached to respective carbon atoms which are separated one from another by a single carbon atom.

Preferably, the polyhydric alcohol used in the thermoplastic composition is pentaerythritol, dipentaerythritol, tripentaerythritol, di-trimethylolpropane, D-mannitol, D-sorbitol and xylitol. More preferably, the polyhydric alcohol used is dipentaerythritol and/or tripentaerythritol. A most preferred polyhydric alcohol is dipentaerythritol.

The thermoplastic composition may comprise 0.1 to 10 weight percent of at least one polyhydroxy polymer having a number average molecular weight (Mₙ) of at least 2000, selected from the group consisting of ethylene/vinyl alcohol copolymers; as determined for polymeric materials with gel permeation chromatography (GPC). Preferably the polyhydroxy polymer has a Mₙ of 5000 to 50,000.

In one embodiment the polyhydroxy polymer is an ethylene/vinyl alcohol copolymer (EVOH). The EVOH may have a vinyl alcohol repeat content of 10 to 90 mol % and preferably 30 to 80 mol %, 40 to 75 mol %, 50 to 75 mol %, and 50 to 60 mol %, wherein the remainder mol % is ethylene. A suitable EVOH for the thermoplastic composition is Soarnol® A or D copolymer available from Nippon Gosei (Tokyo, Japan) and EVAL® copolymers available from Kuraray, Tokyo, Japan.

The thermoplastic composition may comprise 1 to 10 weight percent; and preferably 1 to 7 weight percent and more preferably 2 to 7 weight percent polyhydroxy polymer based on the total weight of the thermoplastic polyamide composition.

The thermoplastic composition may comprise 0 to 3 weight percent of one or more co-stabilizer(s) having a 10% weight loss temperature, as determined by thermogravimetric analysis (TGA), of greater than 30° C. below the melting point of the polyamide resin, if a melting point is present, or at least 250° C. if said melting point is not present, selected from the group consisting of secondary aryl amines, hindered phenols and hindered amine light stabilizers (HALS), and mixtures thereof.

For the purposes of this invention, TGA weight loss will be determined according to ASTM D 3850-94, using a heating rate of 10° C./min, in air purge stream, with an appropriate flow rate of 0.8 mL/second. The one or more co-stabilizer(s) preferably has a 10% weight loss temperature, as determined by TGA, of at least 270° C., and more preferably 290° C., 320° C., and 340° C., and most preferably at least 350° C.

The one or more co-stabilizers preferably are present from at or about 0.1 to at or about 3 weight percent, more preferably at or about 0.2 to at or about 1.2 weight percent; or more preferably from at or about 0.5 to at or about 1.0 weight percent, based on the total weight of the thermoplastic composition.

Secondary aryl amines useful in the invention are high molecular weight organic compound having low volatility. Preferably, the high molecular weight organic compound will be selected from the group consisting of secondary aryl amines further characterized as having a molecular weight of at least 260 g/mol and preferably at least 350 g/mol, together with a 10% weight loss temperature as determined by thermogravimetric analysis (TGA) of at least 290° C., preferably at least 300° C., 320° C., 340° C., and most preferably at least 350° C.

By secondary aryl amine is meant an amine compound that contains two carbon radicals chemically bound to a nitrogen atom where at least one, and preferably both carbon radicals, are aromatic. Preferably, at least one of the aromatic radicals, such as, for example, a phenyl, naphthyl or heteroaromatic group, is substituted with at least one substituent, preferably containing 1 to about 20 carbon atoms.

Examples of suitable secondary aryl amines include 4,4′ di(α,α-dimethylbenzyl)diphenylamine available commercially as Naugard 445 from Uniroyal Chemical Company, Middlebury, Conn.; the secondary aryl amine condensation product of the reaction of diphenylamine with acetone, available commercially as Aminox from Uniroyal Chemical Company; and para-(paratoluenesulfonylamido)diphenylamine also available from Uniroyal Chemical Company as Naugard SA. Other suitable secondary aryl amines include N,N′-di-(2-naphthyl)-p-phenylenediamine, available from ICI Rubber Chemicals, Calcutta, India. Other suitable secondary aryl amines include 4,4′-bis(α,α′-tertiaryoctyl)diphenylamine, 4,4′-bis(ca-methylbenzhydryl)diphenylamine, and others from EP 0509282 B1.

The hindered amine light stabilizers (HALS) may be one or more hindered amine type light stabilizers (HALS). HALS are compounds of the following general formulas and combinations thereof:

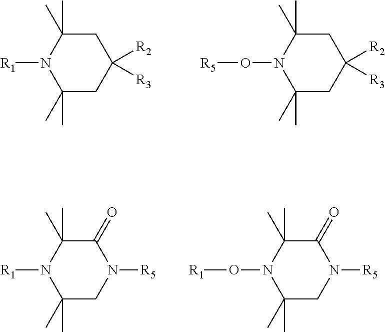

In these formulas, R₁ up to and including R₅ are independent substituents. Examples of suitable substituents are hydrogen, ether groups, ester groups, amine groups, amide groups, alkyl groups, alkenyl groups, alkynyl groups, aralkyl groups, cycloalkyl groups and aryl groups, in which the substituents in turn may contain functional groups; examples of functional groups are alcohols, ketones, anhydrides, imines, siloxanes, ethers, carboxyl groups, aldehydes, esters, amides, imides, amines, nitriles, ethers, urethanes and any combination thereof. A hindered amine light stabilizer may also form part of a polymer or oligomer.

Preferably, the HALS is a compound derived from a substituted piperidine compound, in particular any compound derived from an alkyl-substituted piperidyl, piperidinyl or piperazinone compound, and substituted alkoxypiperidinyl compounds. Examples of such compounds are: 2,2,6,6-tetramethyl-4-piperidone; 2,2,6,6-tetramethyl-4-piperidinol; bis-(1,2,2,6,6-pentamethyl piperidyl)-(3′,5′-di-tert-butyl-4′-hydroxybenzyl) butylmalonate; di-(2,2,6,6-tetramethyl-4-piperidyl) sebacate (Tinuvin® 770, MW 481); oligomer of N-(2-hydroxyethyl)-2,2,6,6-tetramethyl-4-piperidinol and succinic acid (Tinuvin® 622); oligomer of cyanuric acid and N,N-di(2,2,6,6-tetramethyl-4-piperidyl)-hexamethylene diamine; bis-(2,2,6,6-tetramethyl-4-piperidinyl) succinate; bis-(1-octyloxy-2,2,6,6-tetramethyl-4-piperidinyl) sebacate (Tinuvin® 123); bis-(1,2,2,6,6-pentamethyl-4-piperidinyl) sebacate (Tinuvin® 765); Tinuvin® 144; Tinuvin® XT850; tetrakis-(2,2,6,6-tetramethyl-4-piperidyl)-1,2,3,4-butane tetracarboxylate; N,N′-bis-(2,2,6,6-tetramethyl-4-piperidyl)-hexane-1,6-diamine (Chimasorb® T5); N-butyl-2,2,6,6-tetramethyl-4-piperidinarine; 2,2′-[(2,2,6,6-tetramethyl-piperidinyl)-imino]-bis-[ethanol]; poly((6-morpholine-5-triazine-2,4-diyl)(2,2,6,6-tetramethyl-4-piperidinyl)-iminohexarethylene-(2,2,6,6-tetramethyl-4-piperidinyl)-imino) (Cyasorb® UV 3346); 5-(2,2,6,6-tetramethyl-4-piperidinyl)-2-cyclo-undecyl-oxazole) (Hostavin® N20); 1,1′-(1,2-ethane-di-yl)-bis-(3,3′,5,5′-tetramethyl-piperazinone); 8-acetyl-3-dothecyl-7,7,9,9-tetramethyl-1,3,8-triazaspiro(4,5)decane-2,4-dione; polymethylpropyl-3-oxy-[4(2,2,6,6-tetramethyl)-piperidinyl]siloxane (Uvasil® 299); 1,2,3,4-butane-tetracarboxylic acid-1,2,3-tris(1,2,2,6,6-pentamethyl-4-piperidinyl)-4-tridecylester; copolymer of alpha-methylstyrene-N-(2,2,6,6-tetramethyl-4-piperidinyl) maleimide and N-stearyl maleimide; 1,2,3,4-butanetetracarboxylic acid, polymer with beta,beta,beta′,beta′-tetramethyl-2,4,8,10-tetraoxaspiro[5.5]undecane-3,9-diethanol, 1,2,2,6,6-pentamethyl-4-piperidinyl ester (Mark® LA63); 2,4,8,10-tetraoxaspiro[5.5]undecane-3,9-diethanol, beta, beta, beta′, beta′-tetram ethyl-polymer with 1,2,3,4-butanetetracarboxylic acid, 2,2,6,6-tetramethyl-4-piperidinyl ester (Mark® LA68); D-glucitol, 1,3:2,4-bis-O-(2,2,6,6-tetramethyl-4-piperidinylidene)-(HALS 7); oligomer of 7-oxa-3,20-diazadispiro[5.1.11.2]-heneicosan-21-one-2,2,4,4-tetramethyl-20-(oxiranylmethyl) (Hostavin® N30); propanedioic acid, [(4-methoxyphenyl)methylene]-,bis(1,2,2,6,6-pentamethyl-4-piperidinyl) ester (Sanduvor® PR 31); formamide, N,N′-1,6-hexanediylbis[N-(2,2,6,6-tetramethyl-4-piperidinyl (Uvinul® 4050H); 1,3,5-triazine-2,4,6-triarine, N,N′″-[1,2-ethanediylbis[[[4,6-bis[butyl(1,2,2,6,6-pentamethyl-4-piperidinyl)amino]-1,3,5-triazine-2-yl]imino]-3,1-propanediyl]]-bis[N′,N″-dibutyl-N′, N″-bis(1,2,2,6,6-pentamethyl-4-piperidinyl) (Chimassorb® 119 MW 2286); poly[[6-[(1,1,3,33-tetramethylbutyl)amino]-1,3,5-triazine-2,4-diyl][(2,2,6,6-tetramethyl-4-peperidinyl)-imino]-1,6-hexanediyl[(2,2,6,6-tetramethyl-4-piperidinyl)imino]](Chimassorb® 944 MW 2000-3000); 1,5-dioxaspiro(5,5) undecane 3,3-dicarboxylic acid, bis(2,2,6,6-tetramethyl-4-peridinyl) ester (Cyasorb® UV-500); 1,5-dioxaspiro(5,5) undecane 3,3-dicarboxylic acid, bis(1,2,2,6,6-pentamethyl-4-peridinyl) ester (Cyasorb® UV-516); N-2,2,6,6-tetramethyl-4-piperidinyl-N-amino-oxamide; 4-acryloyloxy-1,2,2,6,6-pentamethyl-4-piperidine. 1,5,8,12-tetrakis[2′,4′-bis(1″,2″,2″,6″,6″-pentamethyl-4″-piperidinyl(butyl)amino)-1′,3′,5′-triazine-6′-yl]-1,5,8,12-tetraazadodecane; HALS PB-41 (Clariant Huningue S. A.); Nylostab® S-EED (Clariant Huningue S. A.); 3-dodecyl-1-(2,2,6,6-tetramethyl-4-piperidyl)-pyrrolidin-2,5-dione; Uvasorb® HA88; 1,1′-(1,2-ethane-di-yl)-bis-(3,3′,5,5′-tetra-methyl-piperazinone) (Good-rite® 3034); 1,1,′1″-(1,3,5-triazine-2,4,6-triyltris((cyclohexylimino)-2,1-ethanediyl)tris(3,3,5,5-tetramethylpiperazinone) (Good-rite® 3150) and; 1,1′,1″-(1,3,5-triazine-2,4,6-triyltris((cyclohexylimino)-2,1-ethanediyl)tris(3,3,4,5,5-tetramethylpiperazinone) (Good-rite® 3159). (Tinuvin® and Chimassorb® materials are available from Ciba Specialty Chemicals; Cyasorb® materials are available from Cytec Technology Corp.; Uvasil® materials are available from Great Lakes Chemical Corp.; Saduvor®, Hostavin®, and Nylostab® materials are available from Clariant Corp.; Uvinul® materials are available from BASF; Uvasorb® materials are available from Partecipazioni Industriali; and Good-rite® materials are available from B.F. Goodrich Co. Mark® materials are available from Asahi Denka Co.)

Other specific HALS are selected from the group consisting or di-(2,2,6,6-tetramethyl-4-piperidyl) sebacate (Tinuvin® 770, MW 481) Nylostab® S-EED (Clariant Huningue S. A.); 1,3,5-triazine-2,4,6-triamine, N,N′″-[1,2-ethanediylbis[[[4,6-bis[butyl(1,2,2,6,6-pentamethyl-4-piperidinyl)amino]-1,3,5-triazine-2-yl]imino]-3,1-propanediyl]]-bis[N′,N″-dibutyl-N′,N″-bis(1,2,2,6,6-pentamethyl-4-piperidinyl) (Chimassorb® 119 MW 2286); and poly[[6-[(1,1,3,33-tetramethylbutyl)amino]-1,3,5-triazine-2,4-diyl][(2,2,6,6-tetramethyl-4-peperidinyl)-imino]-1,6-hexanediyl[(2,2,6,6-tetramethyl-4-piperidinyl)imino]](Chimassorb® 944 MW 2000-3000).

Mixtures of secondary aryl amines and HALS may be used. A preferred embodiment comprises at least two co-stabilizers, at least one selected from the secondary aryl amines; and at least one selected from the group of HALS, as disclosed above, wherein the total weight percent of the mixture of co-stabilizers is at least 0.5 wt percent, and preferably at least 0.9 weight percent.

By hindered phenol is meant an organic compound containing at least one phenol group wherein the aromatic moiety is substituted at least at one and preferably at both positions directly adjacent to the carbon having the phenolic hydroxyl group as a substituent. The substituents adjacent the hydroxyl group are alkyl radicals suitably selected from alkyl groups having from 1 to 10 carbon atoms, and preferably will be tertiary butyl groups. The molecular weight of the hindered phenol is suitably at least about 260, preferably at least about 500, more preferably at least about 600. Most preferred are hindered phenols having low volatility, particularly at the processing temperatures employed for molding the formulations, and may be further characterized as having a 10% TGA weight loss temperature of at least 290° C., preferably at least 300° C., 320° C., 340° C., and most preferably at least 350° C.

Suitable hindered phenol compounds include, for example, tetrakis(methylene (3,5-di-(tert)-butyl-4-hydroxyhydrocinnamate)) methane, available commercially as Irganox® 1010 from CIBA Specialty Chemicals, Tarrytown, N.Y. and N,N′-hexamethylene bis(3,5-di-(tert)butyl-hydroxyhydro-cinnamamide) also available from CIBA Specialty Chemicals as Irganox® 1098. Other suitable hindered phenols include 1,3,5-trimethyl-2,4,6-tris(3,5-di-(tert)-butyl-4-hydroxybenzyl)benzene and 1,6hexamethylene bis(3,5-di-(tert)butyl-4-hydroxy hydrocinnamate), both available from CIBA Specialty Chemicals as Irganox® 1330 and 259, respectively. A preferred co-stabilizer for the polyamide composition is a hindered phenol. Irganox 1098 is a most preferred hindered phenol for the compositions.

Mixtures of polyhydric alcohols, secondary aryl amines, hindered phenols, and HALS may be used. A preferred embodiment includes at least one polyhydric alcohol and at least one secondary aryl amine in the weight ranges defined above.

The thermoplastic composition may comprise about 0.1 to at or about 1 weight percent, or more preferably from at or about 0.1 to at or about 0.7 weight percent, based on the total weight of the polyamide composition, of copper salts. Copper halides are mainly used, for example CuI, CuBr, Cu acetate and Cu naphthenate. Cu halides in combination with alkali halides such as KI, KBr or LiBr may be used. Copper salts in combination with at least one other stabilizer selected from the group consisting of poyhydric alcohols, polyhric polymers, secondary aryl amines and HALS; as disclosed above, may be used as thermal stabilizers.

Herein the thermoplastic composition is compounded by a melt-blending method, in which the ingredients are appropriately dispersed in a polymer matrix during the compounding process. Any melt-blending method may be used for mixing the ingredients and the polymeric materials of the present invention. For example, polymeric material and the ingredients may be fed into a melt mixer through a single feeder or multiple feeders of a single screw extruder or twin screw extruder, agitator, kneader, or Banbury mixer, and the addition of all the components may be carried out in a single cycle process or by batch process in a multiple cycles. When the polymeric material and different ingredients are added in batches in multiple cycles, a part of the polymeric material and/or ingredients are first melt blended, and in subsequent stages melt blended products are further melt-mixed with the remaining polymeric materials and/or ingredients until an adequately mixed composition is obtained. If a reinforcing filler presents a long physical shape (for example, a long glass fiber), drawing extrusion molding or pultrusion process may be used to prepare a reinforced composition.

Another embodiment is the use of a polyamide or copolyamide consisting essentially of repeat units selected from the group consisting of formulas structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; wherein at least one repeat unit is selected from structures (I) and (II); and any one the repeat units may be present up to about 92 mole percent; to provide ZnCl₂ salt resistance in injection molded thermoplastic articles. All the embodiments disclosed above for the copolyamide consisting essentially of formula (I) to (IV) are also applicable to their use in providing ZnCl₂ salt resistance.

Another embodiment is the use of a polyamide or copolyamide consisting essentially of repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);

and n is an integer selected from 4, 6, 10 or 12; wherein at least one repeat unit is selected from structures (I) and (I); and any one the repeat units may be present up to about 92 mole percent; wherein rectangular test pieces measuring 50 mm×12 mm×3.2 mm, prepared from said polyamide composition, have a resistance to 50% by weight aqueous solution of ZnCl₂ of at least 24 hours at 50° C., when measured according to ASTM D1693, Condition A, adapted for determining stress cracking resistance of the polyamide compositions as disclosed herein.

In another aspect, the present invention relates to a method for manufacturing an article by shaping the melt-mixed compositions. Examples of articles are films, laminates, filaments, fibers, monolayer tubes, hoses, pipes, multi-layer tubes, hoses and pipes with one or more layers formed from the above composition, and automotive parts including engine parts. By “shaping”, it is meant any shaping technique, such as for example extrusion, injection molding, thermoform molding, compression molding, blow molding, filament spinning, sheet casting or film blowing. Preferably, the article is shaped by extrusion or injection molding.

Another embodiment is a molded or extruded thermoplastic article comprising the thermoplastic composition disclosed above. The molded or extruded thermoplastic articles disclosed herein may have application in many vehicular components that meet one or more of the following requirements: high impact strength; high flexural strength; significant weight reduction (over conventional metals, for instance); resistance to high temperature; resistance to light; resistance to oil; resistance to chemical agents such as coolants and road salts; and noise reduction allowing more compact and integrated design. Specific molded or extruded thermoplastic articles are selected from the group consisting of fasteners; fenders; gears; charge air coolers (CAC); cylinder head covers (CHC); oil pans; engine cooling systems, including thermostat and heater housings and coolant pumps; exhaust systems including mufflers and housings for catalytic converters; air intake manifolds (AIM); and timing chain belt front covers. Other molded or extruded thermoplastic articles disclosed herein are selected from the group consisting of pipes for transporting liquids and gases, inner linings for pipes, fuel lines, air break tubes, coolant pipes, air ducts, pneumatic tubes, hydraulic houses, cable covers, cable ties, connectors, canisters, and push-pull cables.

Another embodiment is a flexible tubing comprising:
(a) a copolyamide comprising at least two repeat units selected from the group consisting of structures (I) to (IV),

—C(O)(CH₂)₁₀C(O)NH(CH₂)ₙNH— (I);

—C(O)(CH₂)₁₂C(O)NH(CH₂)ₙNH— (II);

—C(O)(CH₂)₁₄C(O)NH(CH₂)ₙNH— (II);

and —C(O)(CH₂)₁₆C(O)NH(CH₂)ₙNH— (IV);
and n is an integer selected from 4, 6, 10 or 12; wherein at least one repeat unit is selected from structures (I) and (II); and any one the repeat units may be present up to about 92 mole percent;
(b) 0 to 30 weight percent of at least one polymeric toughener;
(d) 0 to 10 weight percent thermal stabilizer; and
(e) 0 to 20 weight percent of plasticizer; and
wherein the weight percent of (a), (b), and (d) and (e) are based on the total weight of the thermoplastic composition.

The flexible tubing composition may include a sulfonamide plasticizer. Suitable sulfonamide plasticizers include aromatic sulfonamides such as benzenesulfonamides and toluenesulfonamides. Examples of suitable sulfonamides include N-alkyl benzenesulfonamides and toluenesulfonamides, such as N-butylbenzenesulfonamide, N-(2-hydroxypropyl)benzenesulfonamide, N-ethyl-o-toluenesulfonamide, N-ethyl-p-toluenesulfonamide, o-toluenesulfonamide, p-toluenesulfonamide, and the like. Preferred are N-butylbenzenesulfonamide, N-ethyl-o-toluenesulfonamide, and N-ethyl-p-toluenesulfonamide.

Further examples of palsticizers include polyamide oligomers with a number average molecular weight of 800 to 5000 g/mol, as disclosed in U.S. Pat. No. 5,112,908, herein incorporated by reference, and US patent publication 2009/0131674 A1. Preferred polyamide oligomers have an inherent viscosity less than 0.5.

The plasticizer may be incorporated into the flexible tubing composition by melt-blending the copolyamide with plasticizer and, optionally, other ingredients, or during polymerization. If the plasticizer is incorporated during polymerization, the polyamide monomers are blended with one or more plasticizers prior to starting the polymerization cycle and the blend is introduced to the polymerization reactor. Alternatively, the plasticizer can be added to the reactor during the polymerization cycle.

When present, the plasticizer is present in the composition in about 1 to about 20 weight percent, or more preferably in about 6 to about 18 weight percent, or yet more preferably in about 8 to about 15 weight percent, wherein the weight percentages are based on the total weight of the composition.

The present invention is further illustrated by the following examples. It should be understood that the following examples are for illustration purposes only, and are not used to limit the present invention thereto.

### Methods

### % Biobased Carbon

ASTM-D6866 Method B Biobased Determination method were conducted by Beta Analytics Inc. Miami Fla., USA, to determine the % biobased carbon.

Data in Tables 2 and 4 was Obtained with the Following Methods:

### Melting Points

Melting points and glass transition temperatures were measured on a TA Instruments DSC 2910 using ASTM Method ASTM D3418 at a heating rate of 10° C./min. On the second heat the melting point is taken as the peak of the melting endotherm, and the glass transition temperature is taken at the transition midpoint.

### Melt Viscosity

Melt viscosity was measured on a Dynisco LCR 7001 at 280 C and 1000 sec-1 shear rate.

### Physical Properties Measurement

Mechanical tensile properties: Tensile strength (TS, stress at break) and elongation at break (EB, strain at break) were measured according to ISO 527-2/1A. Measurements were made on injection molded ISO tensile bar with melt temperature at 295-300° C.; mold temperature at 100° C. and a hold pressure of 85 MPa, with a thickness of the test specimen of 4 mm and a width of 10 mm according to ISO 527/1A at a testing speed of 5 mm/min (tensile strength and elongation) and 50 mm/min for unreinforced samples.

Flexural modulus and flexural strength were measured per ISO 178.

Notched Charpy was measured per ISO 179.

Data in Table 3 was Obtained with the Following Methods:

### Melting Point

Herein melting points were as determined with DSC at a scan rate of 10° C./min in the first heating scan, wherein the melting point is taken at the maximum of the endothermic peak.

### Freezing Point

Herein freezing points were as determined with DSC at a scan rate of 10° C./min in the cooling cycle as per ASTM D3418.

### Inherent Viscosity

Inherent viscosity (IV) was measured on a 0.5% solution of copolyamide in m-cresol at 25° C.

### Mechanical Properties

Polyamides obtained from single preparation batches or multiple preparation batches (2 to 3 batches) were cube blended, dried and then injection molded into test bars. The tensile and flexural properties listed in Table 3 were measured as per ASTM D638 and ASTM D790 test procedures, respectively. Yield stress and Young's modulus were measured using 115 mm (4.5 in) long and 3.2 mm (0.13 in) thick type IV tensile bars per ASTM D638-02a test procedure with a crosshead speed of 50 mm/min (2 in/min). Flexural modulus was measured using 3.2 mm (0.13 in) thick test pieces per ASTM D790 test procedure with a 50 mm (2 in) span, 5 mm (0.2 in) load and support nose radii and 1.3 mm/min (0.05 in/min) crosshead speed.

### DMA Test Method

Dynamic mechanical analysis (DMA) test was done using TA instruments DMA Q800 equipment. Injection molded test bars nominally measuring 18 mm×12.5 mm×3.2 mm were used in single cantilever mode by clamping their one end. The bars were equilibrated to −140° C. for 3 to 5 minutes, and then DMA test was carried out with following conditions: temperature ramping up from −140° C. to +150° C. at a rate of 2 degrees C./min, sinusoidal mechanical vibration imposed at an amplitude of 20 micrometers and multiple frequencies of 100, 50, 20, 10, 5, 3 and 1 Hz with response at 1 Hz selected for determination of storage modulus (E′) and loss modulus (E″) as a function of temperature. Tan delta was computed by dividing the loss modulus (E″) by the storage modulus (E′).

### Zinc Chloride Resistance Test

ASTM D1693, Condition A, provides a test method for determination of environmental stress-cracking of ethylene plastics in presence of surface active agents such as soaps, oils, detergents etc. This procedure was adapted for determining stress cracking resistance of the polyamide compositions to a 50% by weight aqueous solution of ZnCl₂ as follows.

Rectangular test pieces measuring 37.5 mm×12 mm×3.2 mm were molded. A controlled nick was cut into the face of each molded bar as per the standard procedure, the bars were bent into U-shape with the nick facing outward, and positioned into brass specimen holders as per the standard procedure. At least five bars were used for each composition. The holders were positioned into large test tubes.

The test fluid used was 50 wt % zinc chloride solution prepared by dissolving anhydrous zinc chloride into water in 50:50 weight ratio. The test tubes containing specimen holders were filled with freshly prepared salt solution fully immersing the test pieces such that there was at least 12 mm of fluid above the top test piece. The test tubes were positioned upright in a circulating air oven maintained at 50° C. Test pieces were periodically examined for development of cracks over a period of either 24 hours of immersion followed by 24 hrs of dry-out under ambient conditions without wiping or continued immersion of up to 200 hours as indicated in the tables below. Time to first observation of failure in any of the test pieces was recorded. After 191 hours of continued immersion, test pieces were withdrawn from the zinc chloride solution and without wiping, dried in an oven at 50° C. for another 24 hours. Time to first observation of failure in any of the test pieces was recorded.

### Materials

Several mixtures of linear alkanes were provided by the hydrotreating process is disclosed in WO 2010/068904.

### Hydrotreating of Palm Oil

Refined Palm oil (50 g manufactured by T.I. International Ghana Ltd., of Accra, Ghana) and alumina supported pre-sulfided cobalt/nickel/molybdenum trimetallic hydrotreating catalyst (5 g, CRI DC2318, commercially available from Criterion Catalysts and Technologies, Houston Tex.) were placed in a 210 cc agitated pressure reactor. The vessel was leaked check with nitrogen. The headspace of the reactor was purged with nitrogen 10 times by pressurizing to 90 psig (722 kPa) and depressurizing to 0 psig (101 kPa). The reactor was then purged with high purity hydrogen (99.9% min., Commercially available from Air Products, Allentown Pa.) five times and pressurized to 1000 psig (7000 kPa) with hydrogen. The reactor and its contents were assigned were agitated and heated to 325° C. (617° F.). The hydrogen pressure was increased to 2000 psig (13, 900 kPa), and maintained there for 5 hours. The headspace was filled with fresh hydrogen to 1500-1700 psig (11,800 kPa) if the pressure dropped below 1000 psig (7000 kPa).

The reactor contents were then cooled to below 50° C. (122° F.), the headspace was vented, and the contents were discharged to a glass bottle.

The content were weighed. IR and 1H NMR analysis showed no evidence of mono, di-, and triacylglycerides. The reaction products were analyzed by GC-FID to obtain the following linear hydrocarbon distribution by weight: C18+, =0.5%, C18=46.5%, C₁₇=5%, C16=43%, C15=4%, C14=1%.

The mixture of linear hydrocarbon is directly used for fermentation without isolation or purification.

### Hydroteating Palm Kernel Oil

The process of example 1 was repeated using the same equipments, pressure and temperature except 10 g of the same CRI DC-2318 catalyst and palm kernel oil (100 g, obtained from Columbus Foods Company Des Plains Ill. via their web based division soaperchoice.com) were used in a larger (400 cc) agitated pressure reactor. The reaction products were analyzed by GC-FID to obtain the following linear hydrocarbon distribution by weight: C₁₈₊=0.4%, C₁₈=19.3%, C₁₇=2.5%, C₁₆=7.9%, C₁₅=1.1%, C₁₄=15.0%, C₁₃=2.0%, C₁₂=40.2%, C₁₁=4.6%, C₁₀=3.0%, C₉=0.4%, C₈=3.2%, C₈₋=0.4%. The mixture of linear hydrocarbon is directly used for fermentation without isolation or purification.

### Hydrotreating Soybean Oil

The process of example 1 was repeated using the same equipments, pressure, temperature and catalyst (5 g), except soybean oil (50 g, obtained from Sigma-Aldrich of St. Louis, Mo.) was used. The content were weighed. IR and ¹H NMR analysis showed no evidence of mono, di-, and triacylglycerides. The reaction products were analyzed by GC-FID to obtain the following linear hydrocarbon distribution by weight: C₁₈₊=1%, C₁₈=81%, C₁₇=6%, C₁₆=11.5%, C₁₅=0.5%, C₁₄=15.0%. The mixture of linear hydrocarbon is directly used for fermentation without isolation or purification.

### Hydrotreating Chicken Fat

The process of example 1 was repeated using the same equipments, pressure, temperature and catalyst (5 g), except chicken fat (50 g, obtained from Perdue Farms Salisbury Md.) was used. The reaction products were analyzed by GC-FID to obtain the following linear hydrocarbon distribution by weight: C₁₈₊=1%, C₁₈=60%, C₁₇=7%, C₁₆=28%, C₁₅=3%, C₁₄=1%. The mixture of linear hydrocarbon is directly used for fermentation without isolation or purification.

### Hydrotreating 50/50 Chicken Fat/Soybean Oil

The process of example 1 was repeated using the same equipments, pressure, temperature and catalyst (5 g), except a 50:50 chicken fat to soybean oil (50 g, mixed in house with chicken fat and soybean oil obtained from Perdue Farms Salisbury Md.) was used. The reaction products were analyzed by GC-FID to obtain the following linear hydrocarbon distribution by weight: C₁₈₊=2.1%, C₁₈=69.6%, C₁₇₌7.2%, C₁₆=18.5%, C₁₅=1.9%, C₁₄=0.5%, C₁₃=0.1%, C_{12=0.1}%. The mixture of linear hydrocarbon is directly used for fermentation without isolation or purification.

PA610 refers to Zytel® RS3090 polyamide 610 made from 1,6-diaminohexane and 1,10-decanedioic acid having a melting point of 224° C., available from E.I. DuPont de Nemours and Company, Wilmington, Del., USA.

PA612 refers to Zytel® 158 NC010 resin, having a melting point of about 218° C., available from E. I. du Pont de Nemours and Company, Wilmington, Del.

Uniplex® 214 plasticizer, refers to N-butylbenzenesulfonamide available from Unitex Chemical Corp., Greensboro, N.C.

Glass fibers C refers to CPIC 301HP chopped glass fiber available from Chongqing Polycomp International Corp., Chongqing, China.

Glass Fiber E refers to PPG 3660 chopped glass fiber available fro PPG Industries, Pittsburgh, Pa.

TRX®301 copolymer refers to a maleic anhydride modified EPDM from available from E.I. DuPont de Nemours and Company, Wilmington, Del., USA.

Engage® 8180 refers to an ethylene-octene copolymer commercially available Dow Chemical Co., Midland, Mich.

Fusabond® N598 is a maleic anhydride grafted ethylene copolymer, available from E.I. DuPont de Nemours and Company, Wilmington, Del., USA.

Licomont® CaV 102 fine grain is calcium salt of montanic acid available from Clariant Corp., 4132 Mattenz, Switzerland.

Cu heat stabilizer refers to a mixture of 7 parts of potassium iodide and 1 part of copper iodide in 1 part of a stearate wax binder.

Naugard® 445 stabilizer refers to 4,4′ di(.α,α-dimethylbenzyl)diphenylamine available commercially from Chemtura Chemical Company, Middlebury, Conn.

Lowinox 44B25 is an antioxidant obtained from Great Lakes Chemical Corporation.

Irgafos® 168 stabilizer is a phosphite anti-oxidant from BASF.

Carbon black refers to ZYTEMCB32 BKO35C black master batch consisting of 45 weight percent carbon black in an ethylene/methacrylic acid copolymer available from Ampacet Corporation, Tarrytown, N.Y.

Lubricant is aluminum distearate

### Example 1

Production of a mixture of dodecanedioic acid and tetradecanedioic acid from a mixture of the corresponding chain length, C12 and C14, alkanes by Candida maltosa ATCC 74430 (prophetic):

A 10 ml, seed inoculum of Candida maltosa strain ATCC 74430 is grown for 24 h at 30° C. with shaking at 250 rpm in a solution containing 10 g/L yeast extract+20 g/L peptone+20 g/L glucose. The resulting cell suspension is inoculated into 2×350 mL of pH 5 yeast minimal medium consisting of 3 g/L (NH4)2SO4, 6.6 g/L KH2PO4, 0.4 g/L K2HP04, 0.6 g/L anhydrous MgS04, 4 g/L yeast extract, 75 g/L glucose, 100 mg/l, biotin, 13 mg/L FeS04-7H2O, 2 mg/L CuS04-51-120, 20 mg/L ZnS04-7H20, 6 mg/L MnS04-H20, 2 mg/L Co(N03)2.6H20, 3 mg/L NaMo04-2H20 and 1.6 mg/L KI and grown for 24 h at 30° C. with shaking at 250 rpm. A fermenter (Braun) containing 7 L of pH 5 yeast minimal medium is then inoculated with 525 mL of the overnight culture. The fermenter is maintained at minimal airflow and agitation until dissolved oxygen reaches 20% of atmospheric. The dissolved oxygen is then raised to approximately 80% of atmospheric and maintained through fermenter control of aeration up to 2 vvm and agitation up to 1400 rpm at 30° C. The addition of 10% w/v NH40H provided nitrogen for cell growth and also maintained the pH of the medium at 5. After approximately 18 h, glucose concentration reaches approximately zero. The alkane mixture is then added to a final concentration of approximately 20 g/L. The pH of the medium is then adjusted to 7.5 through the addition of 20% w/v KOH. Further additions of 20% w/v KOH maintain pH of the medium at 7.5 for the remainder of the fermentation. The alkane mixture is maintained above 3 g/L in the fermenter. In addition, glucose is fed at a slow rate in the range of 0.2 to 0.8 g glucose/min and glucose concentration is monitored and addition rate is adjusted to keep the glucose concentration below 1 g glucose/L. Approximately 51 h after alkane addition, material from the fermenter is harvested and analyzed for concentrations of dicarboxylic acids.

The dicarboxylic acid mixture is recovered from the whole fermenter liquor (cells and supernatant) by acidifying the liquor to pH 2 with 2M phosphoric acid and extracting the precipitated material into 3×5 mL, methyl-tertiary butyl ether. A portion of the ether extract is evaporated to dryness and the recovered dicarboxylic acid mixture is analyzed as a MSTFA (N-methyl-N-trimethylsilyltrifluoroacetamide) derivative and analyzed by gas chromatography by methods known in the art.

Material recovered from the fermentation consists of a mixed diacid product. The C12 diacid is present at 10 g/L or a total yield of 80 g from the fermenter. The C14 diacid product is present at 6 g/L or a total yield of 48 g from the fermenter.

### Example 2

Production of a mixture of hexadecanedioic acid and octadecanedioic acid from a mixture of the corresponding chain length, C16 and C18, alkanes by Candida tropicalis CGMCC NO. 0206 (Center of General Microbiology of China Committee for Culture Collection of Microorganisms) (prophetic):

A seed culture of Candida tropicalis CGMCC 0206 is grown up in 25 ml of alkane seed medium: tap water with KH2PO4, 8 g/L, yeast extract, 5 g/L, corn extract, 3 g/L, sucrose, 5 g/L, urea 3 g/L, n-hexadecane 70 mL, pH 5.0. Growth occurs at 30° C. on a rotating shaker at 220 rpm for 48 hours. This inoculum is transferred to 500 mL of the same medium and grown under the same conditions for an additional 24 hours

From the seed growth 500 ml of the seed suspension is added to a 10 L fermenter containing 7 L of fermentation medium: KH2PO4, 8 g/L, corn extract, 1 g/L, NaCl, 1.5 g/L, urea, 1 g/L, alkane mixture, 70 g/L, anti-foam, 500 ppm, KN03 6 g/L, dissolved with tap water, pH 7.5 The fermentation is run at 30° C. with oxygen levels maintained at 20% of atmospheric for 4 days. A 20% NaOH solution is added periodically to adjust pH within 7.5-8. Further additions of 20% w/v KOH maintain pH of the medium at 7.5 for the remainder of the fermentation. The alkane mixture is maintained above 10 g/L in the fermenter by periodic additions

The dicarboxylic acid mixture is recovered from the whole fermenter liquor (cells and supernatant) by acidifying the liquor to pH 2 with 2M phosphoric acid and extracting the precipitated material into 3×5 mL, methyl-tertiary butyl ether. A portion of the ether extract is evaporated to dryness and the recovered dicarboxylic acid mixture is analyzed as a MSTFA (N-methyl-N-trimethylsilyltrifluoroacetamide) derivative and analyzed by gas chromatography by methods known in the art.

Material recovered from the fermentation consists of a mixed diacid product. The C16 diacid is present at 25 g/L or a total yield of 200 g from the fermenter. The C18 diacid product is present at 20 g/L or a total yield of 160 g from the fermenter.

### Example 3

Example 3 illustrates the synthesis of PA 612/614 (70/30)

Salt Preparation: PA 612/614 salt solution of approximately 40% by weight in water was prepared as follows: Dodecanedioic acid (34.1 lbs), tetradecanedioic acid (16.38 lbs), an aqueous solution containing about 80 weight % of hexamethylene diamine (HMD) (24.2 lbs dry basis) and water (120 lbs) were added to a salt reactor. The salt solution was heated to 90° C. After complete dissolution, the salt solution was adjusted to a pH of 7.6±0.04. After adjusting to the pH, glacial acetic acid (66.3 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was vented to maintain the pressure at 265 psia. The temperature of the contents was allowed to reach to 248° C. and held for 60 minutes. The pressure was then reduced to about 13 psia over about 20 minutes. The autoclave was then pressurized with nitrogen and the molten polymer was extruded into strands after quenching with cold water and pelletized.

The co-polyamide obtained had an inherent viscosity (IV) of 0.96 dl/g. The polymer had a melting point of 206° C., as measured by DSC.

A second batch of polymer was made with this recipe and the two batches combined for subsequent testing listed in Table 2.

### Example 4

Example 4 illustrates the synthesis of PA 612/614 (80/20)

Salt Preparation: PA 612/614 salt solution of approximately 40% by weight in water was prepared as follows: Dodecanedioic acid (39.3 lbs), tetradecanedioic acid (11.0 lbs), an aqueous solution containing ˜80 weight % of hexamethylene diamine (HMD) (24.7 lbs dry basis) and water (120 lbs) were added to a salt reactor. The salt solution was heated to 90° C. After complete dissolution, the salt solution was adjusted to a pH of 7.6±0.04. After adjusting to the pH of 7.6, glacial acetic acid (66.3 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was

vented to maintain the pressure at 265 psia. The temperature of the contents was allowed to rise to 248° C. and held for 60 minutes. The pressure was then reduced to about 13 psia over about 20 minutes. The autoclave was then pressurized with nitrogen and the molten polymer was extruded into strands, quenched with cold water and cut into pellets.

The co-polyamide obtained had an inherent viscosity (IV) of 0.98 dl/g. The polymer had a melting point of 209° C., as measured by DSC.

### Example 5

Example 5 illustrates the synthesis of PA 612/614/616 (65/25/10)

Salt Preparation Nylon 612/614/616 salt solution of approximately 40% by weight in water was prepared as follows: Dodecanedioic acid (31.28 lbs), tetradecanedioic acid (13.49 lbs), hexadecanedioic acid (6 lbs) an aqueous solution containing about 80 weight % of hexamethylene diamine (HMD) (24.2 lbs dry basis) and water (120 lbs) were added to a salt reactor. The salt solution was heated to 90° C. After complete dissolution, the salt solution was adjusted to a pH of 7.6±0.04. After adjusting to the aim pH, glacial acetic acid (66.3 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was vented to maintain the pressure at 265 psia. The temperature of the contents was allowed to rise to 248° C. and held for 60 minutes. The pressure was then reduced to about 13 psia over about 20 minutes. The autoclave was then pressurized with nitrogen and the molten polymer was extruded into strands, quenched with cold water and cut into pellets.

The ter-polyamide obtained had an inherent viscosity (IV) of 0.93 dl/g. The polymer had a melting point of 202° C., as measured by DSC.

A second batch of polymer was made with this recipe and the two batches combined for subsequent testing.

### Example 6

Example 6 illustrates the synthesis of PA 612/614/616 (60/25/15) Salt Preparation Nylon 612/614/616 salt solution of approximately 40% by weight in water was prepared as follows: Dodecanedioic acid (28.65 lbs), tetradecanedioic acid (13.39 lbs), hexadecanedioic acid (8.9 lbs) an aqueous solution containing about 80 weight % of hexamethylene diamine (HMD) (24.1 lbs dry basis) and water (120 lbs) were added to a salt reactor. The salt solution was heated to 90° C. After complete dissolution, the salt solution was adjusted to a pH of 7.6±0.04. After adjusting to pH of 7.6, glacial acetic acid (56.1 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was vented to maintain the pressure at 265 psia. The temperature of the contents was allowed to rise to 248° C. and held for 60 minutes. The pressure was then reduced to about 13 psia over about 20 minutes. The autoclave was then pressurized with nitrogen and the molten polymer was extruded into strands, quenched with cold water and cut into pellets.

The ter-polyamide obtained had an inherent viscosity (IV) of 0.91 dl/g. The polymer had a melting point of 198° C., as measured by DSC.

### Example 7

Example 12 illustrates the synthesis of PA 612/614/616 (65/20/15)

Salt Preparation: PA 612/614/616 salt solution of approximately 40% by weight in water was prepared as follows: Dodecanedioic acid (31.16 lbs), tetradecanedioic acid (10.75 lbs), hexadecanedioic acid (8.93 lbs) an aqueous solution containing about 80 weight % of hexamethylene diamine (HMD) (24.1 lbs dry basis) and water (120 lbs) were added to a salt reactor. The salt solution was heated to 90° C. After complete dissolution, the salt solution was adjusted to a pH of 7.6±0.04. After adjusting to pH of 7.6, glacial acetic acid (56.1 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was vented to maintain the pressure at 265 psia. The temperature of the contents was allowed to rise to 248° C. and held for 60 minutes. The pressure was then reduced to about 13 psia over about 20 minutes. The autoclave was then pressurized with nitrogen and the molten polymer was extruded into strands, quenched with cold water and cut into pellets.

The terpolyamide obtained had an inherent viscosity (IV) of 0.92 dl/g. The polymer had a melting point of 201° C., as measured by DSC.

### Comparative Example C1

The polyamide PA 612 was prepared by the following process:

Salt Preparation Nylon 612 salt solution of approximately 40% by weight in water was prepared as follows: Dodecanedioic acid (53.2 lbs), an aqueous solution of −80 wt % hexamethylene diamine (HMD) (26.8 lbs dry basis) and water (120 lbs) were added to a salt reactor. The salt solution was heated to 90° C. After complete dissolution, the salt solution was adjusted to a pH of 7.6±0.04. After adjusting to the aim pH, glacial acetic acid (56.3 g) and Carbowax 8000 (0.7 g) were added to the salt tank. The salt solution was then charged to the autoclave.

The autoclave agitator was set to 10 rpm. The agitator was maintained at 10 rpm, the pressure control valve was set to 265 psia, and the autoclave was heated. The pressure was allowed to rise to 265 psia at which point steam was

vented to maintain the pressure at 265 psia. The temperature of the contents was allowed to rise to 248° C. and held for 60 minutes. The pressure was then reduced to −13 psia over about 20 minutes. The autoclave was then pressurized with nitrogen and the molten polymer was extruded into strands, quenched with cold water and cut into pellets.

A second batch of polymer was produced with this recipe and the two batches combined for subsequent testing.

TABLE 2
Polymers of Examples and Comparative Examples

Example

| | C1 | 3 | 4 | 5 | 6 | 7 |
|---|---|---|---|---|---|---|
| Polymer | PA612 | PA612/ | PA612/ | PA612/ | PA612/ | PA612/ |
| | | 614 | 614 | 614/616 | 614/616 | 614/616 |
| | | (70/30) | (80/20) | (65/25/10) | (60/25/15) | (65/20/15) |

DSC data

| | | | | | | |
|---|---|---|---|---|---|---|
| Melting | 218 | 206 | 209 | 202 | 198 | 201 |
| point | | | | | | |
| (° C.) | | | | | | |
| Freezing | 188 | 176 | 179 | 170 | 168 | 169 |
| point | | | | | | |
| (° C.) | | | | | | |
| Tg (° C.) | 44 | 34 | 40 | 37 | 30 | 33 |

Properties

| | | | | | | |
|---|---|---|---|---|---|---|
| MV | 31 | 32 | 29 | 24 | 17 | 20 |
| (Poise) | | | | | | |
| TS, 23° C. | 63 | 49 | 40 | 46 | 34 | 43 |
| (MPa) | | | | | | |
| Flex mod | 2400 | 2050 | 2060 | 1950 | 1840 | 1940 |
| (MPa) | | | | | | |
| N-charpy, | 3.2 | 4.3 | 4.2 | 4.3 | 4.4 | 3.8 |
| 23° C. | | | | | | |
| (KJ/m2) | | | | | | |

### Example 8

Example 8 illustrates the synthesis of PA 614/616 (50/50)

Salt Preparation and polymerization: A 10 L autoclave was charged with tetradecanedioic acid (1189 g), hexadecanedoic acid (1317 g), an aqueous solution containing 78.4 weight % of hexamethylene diamine (HMD) (1374 g), an aqueous solution containing 28 weight percent acetic acid (14 g), an aqueous solution containing 1 weight percent sodium hypophosphite (33 g), an aqueous solution containing 1 weight percent Carbowax 8000 (10 g), and water (2620 g). The process conditions were the same as that described below for PA614.

The co-polyamide obtained had an inherent viscosity (IV) of 1.04 dl/g. The polymer had a melting point of 185° C., as measured by DSC.

### Example 9

Example 9 illustrates the synthesis of PA 614/616 (70/30)

Salt Preparation and polymerization: A 10 L autoclave was charged with tetradecanedioic acid (1688 g), hexadecanedoic acid (802 g) an aqueous solution containing 78.4 weight % of hexamethylene diamine (HMD) (1394 g), an aqueous solution containing 28 weight percent acetic acid (14 g), an aqueous solution containing 1 weight percent sodium hypophosphite (33 g), an aqueous solution containing 1 weight percent Carbowax 8000 (10 g), and water (2615 g). The process conditions were the same as that described above for PA614.

The co-polyamide obtained had an inherent viscosity (IV) of 1.04 dl/g. The polymer had a melting point of 200° C., as measured by differential scanning calorimetry (DSC).

### Comparative Example C2

The polyamide PA 614 was prepared by the following process:

Salt Preparation: A 10 L autoclave was charged with tetradecanedioic acid (2690 g), an aqueous solution containing 78.4 weight % of hexamethylene diamine (HMD) (1554 g), an aqueous solution containing 28 weight percent acetic acid (30 g), an aqueous solution containing 1 weight percent sodium hypophosphite (35 g), an aqueous solution containing 1 weight percent Carbowax 8000 (10 g), and water (2260 g).

Process Conditions The autoclave agitator was set to 5 rpm and the contents were purged with nitrogen at 10 psi for 10 minutes. The agitator was then set to 50 rpm, the pressure control valve was set to 1.72 MPa (250 psi), and the autoclave was heated. The pressure was allowed to rise to 1.72 MPa at which point steam was vented to maintain the pressure at 1.72 Mpa. The temperature of the contents was allowed to rise to 240° C. The pressure was then reduced to 0 psig over about 45 minutes. During this time, the temperature of the contents rose to 255° C. The autoclave pressure was reduced to 5 psia by applying vacuum and held there for 20 minutes. The autoclave was then pressurized with 65 psia nitrogen and the molten polymer was extruded into strands, quenched with cold water and cut into pellets.

### Comparative Example C3

The polyamide PA 616 was prepared by the following process:

Salt Preparation and polymerization: A 10 L autoclave was charged with hexadecanedioic acid (2543 g), an aqueous solution containing 78.4 weight % of hexamethylene diamine (HMD) (1327 g), an aqueous solution containing 28 weight percent acetic acid (14 g), an aqueous solution containing 1 weight percent sodium hypophosphite (33 g), an aqueous solution containing 1 weight percent Carbowax 8000 (10 g), and water (2630 g). The process conditions were the same as that described above for PA614.

The autoclave agitator was set to 10 rpm. The agitator was maintained at 10 rpm, the pressure control valve was set to 265 psia, and the autoclave was heated. The pressure was allowed to rise to 265 psia at which point steam was vented to maintain the pressure at 265 psia. The temperature of the contents was allowed to rise to 248° C. and held for 60 minutes. The pressure was then reduced to ˜13 psia over about 20 minutes. The autoclave was then pressurized with nitrogen and the molten polymer was extruded into strands, quenched with cold water and cut into pellets.

TABLE 3
Properties of PA614, PA616 versus PA614/616 copolyamides

| Example | C2 | C3 | 8 | 9 |
|---|---|---|---|---|
| Polymer Type | PA614 | PA616 | PA614/616 | PA614/616 |
| (Composition) | | | (50/50) | (70/30) |

DSC data

| | | | | |
|---|---|---|---|---|
| Melting point | 213 | 207 | 185 | 200 |
| (° C.) | | | | |
| Heat of fusion | 62 | 65 | 59 | 61 |
| (J/g) | | | | |
| Freezing | 179 | 180 | 166 | 172 |
| point (° C.) | | | | |
| Delta T (MP - | 34.2 | 27 | 19 | 28 |
| FP) (° C.) | | | | |

DMA data

| | | | | |
|---|---|---|---|---|
| Storage | 1781 | 1473 | 1446 | 1431 |
| modulus, | | | | |
| 23° C. (MPa) | | | | |
| Storage | 323 | 280 | 173 | 215 |
| modulus, | | | | |
| 125° C. (MPa) | | | | |
| Tan delta | 59 | 60 | 53 | 56 |
| Tan delta | 0.11 | 0.11 | 0.13 | 0.12 |
| peak value | | | | |

Mechanical properties

| | | | | |
|---|---|---|---|---|
| TS, 23° C. | 52 | 50 | 49 | 48 |
| (MPa) | | | | |
| Flex Modulus | 1938 | 1781 | 1660 | 1609 |
| (Mpa) | | | | |
| TM, 23° C. | 1805 | 1697 | 1578 | 1513 |
| (MPa) | | | | |
| TM, 125° C. | 267 | 243 | 162 | 199 |
| (MPa) | | | | |

Salt stress crack resistance

| | | | | |
|---|---|---|---|---|
| Hours to | >95 | 191 + | >95 | >95 |
| failure at | | 24 hours | | |
| 50° C. | | dry out | | |

### Examples 10-14 and Comparative Example C4

Examples 10-14 illustrate thermoplastic compositions including a reinforcing agent. The compositions and properties are listed in Table 4.

The compositions of Table 4 were prepared by melt blending the formulation ingredients in a Werner & Pfleiderer ZSK 30 operating at about 280° C. using a screw speed of about 300 rpm, a throughput of about 30 lbs/hour and a melt temperature measured by hand of about 290° C. The glass fibers were added to the melt through a screw side feeder. Ingredient quantities shown in Table 2 are given in weight percent on the basis of the total weight of the thermoplastic composition.

TABLE 4
Examples and Comparative Examples of Thermoplastic Compositions.

| | Example | | | | | |
|---|---|---|---|---|---|---|
| | C4 | 10 | 11 | 12 | 13 | 14 |
| PA612 | 66.9 | | | | | |
| PA612/614 | | 66.9 | | | | |
| (70/30) | | | | | | |
| PA612/614 | | | 66.9 | | | |
| (80/20) | | | | | | |
| PA612/614/616 | | | | 66.9 | | |
| (65/25/10) | | | | | | |
| PA612/614/616 | | | | | 66.9 | |
| (60/25/15) | | | | | | |
| PA612/614/616 | | | | | | 66.9 |
| (65/20/15) | | | | | | |
| Glass fiber C | 33 | 33 | 33 | 33 | 33 | 33 |
| lubricant | 0.1 | 0.1 | 0.1 | 0.1 | 0.1 | 0.1 |

Properties

| | | | | | | |
|---|---|---|---|---|---|---|
| TS, 23° C. | 188 | 172 | 177 | 173 | 168 | 173 |
| (MPa) | | | | | | |
| Flexural | 9290 | 8850 | 8800 | 8480 | 8460 | 8780 |
| modulus (MPa) | | | | | | |

### Examples 15-18 and Comparative Examples C5-C10

### Table 5

### Compounding & Molding

Materials were compounded with a 26 mm 13-barrel twin screw extruders at 250 RPM screw speed, 40 pounds per hour throughput, and barrel temperature setting of 250˜270° C. All ingredients were fed from the back of the extruder except the chopped glass fibers which were fed from side of the extruder.

The compounded pellets were dried and molded into 4 mm ISO multipurpose tensile bars on a Nissei Injection Molding Machine FN3000 with barrel temperature setting of 260˜270° C. and with a general compression screw.

### Test Methods Used in Table 5

Tensile strength, elongation at break, and tensile modulus were tested on a tensile tester from Instru-Met Corporation by ISO 527-1/2 at 23° C. and strain rate of 5 mm/min on samples that were dry as molded.

Notched izod was tested on a CEAST impact Tester by ISO 180 at 23° C. on a Type 1A multipurpose specimen with the end tabs cut off. The resulting test sample measures 80×10×4 mm. (The depth under the notch of the specimen is 8 mm). Specimen were dry as molded.

Un-notched izod was tested on a CEAST Impact Tester by ISO 180 at 23° C. on a Type 1A multipurpose specimen with the end tabs cut off. The resulting test sample measures 80×10×4 mm. Specimen were dry as molded.

TABLE 5

| | Example | | | | | | | | | |
|---|---|---|---|---|---|---|---|---|---|---|
| | C5 | C6 | C7 | C8 | C9 | C10 | 15 | 16 | 17 | 18 |
| PA 612 | 59.35 | 49.96 | 0 | 0 | 0 | 0 | 0 | 0 | 0 | 0 |
| PA614 | 0 | 0 | 59.35 | 49.96 | 0 | 0 | 0 | 0 | 0 | 0 |
| PA616 | 0 | 0 | 0 | 0 | 59.35 | 49.96 | 0 | 0 | 0 | 0 |
| PA612/614 (70/30) | 0 | 0 | 0 | 0 | 0 | 0 | 59.35 | 49.96 | 0 | 0 |
| PA614/616 (70/30) | 0 | 0 | 0 | 0 | 0 | 0 | 0 | 0 | 59.35 | 49.96 |
| TRX-301 | 0 | 3.75 | 0 | 3.75 | 0 | 3.75 | 0 | 3.75 | 0 | 3.75 |
| Engage 8180 | 0 | 5.64 | 0 | 5.64 | 0 | 5.64 | 0 | 5.64 | 0 | 5.64 |
| Triblend 7:1:1 | 0.4 | 0.4 | 0.4 | 0.4 | 0.4 | 0.4 | 0.4 | 0.4 | 0.4 | 0.4 |
| Licomont ® | 0.25 | 0.25 | 0.25 | 025 | 0.25 | 0.25 | 0.25 | 0.25 | 0.25 | 0.25 |
| CaV 102 | | | | | | | | | | |
| Glass fiber E | 40 | 40 | 40 | 40 | 40 | 40 | 40 | 40 | 40 | 40 |
| Tensile Strength | 170 | 141 | 160 | 145 | 146 | 135 | 161 | 140 | 147 | 137 |
| (Mpa) | | | | | | | | | | |
| Elongation (%) | 2.66 | 4.06 | 2.39 | 5.44 | 2.32 | 7.09 | 2.5 | 6.04 | 2.28 | 6.03 |
| Tensile Modulus | 11750 | 10427 | 11419 | 10467 | 10818 | 9754 | 11181 | 10507 | 10556 | 10100 |
| (MPa) | | | | | | | | | | |
| Notched Izod | 14.5 | 27.6 | 13.7 | 28.6 | 12.9 | 33.2 | 13.2 | 28.7 | 11.5 | 26 |
| (KJ/m2) | | | | | | | | | | |
| Unnotched Izod | 63.4 | 89 | 57.7 | 86 | 55.3 | 95 | 59.3 | 92.3 | 54 | 84.8 |
| (KJ/m2) | | | | | | | | | | |

### Examples 19, 20 and Comparative Examples C11 to C12

### Table 6

### Compounding and Molding

Compounding was performed in a 25 mm W&P twin screw extruder with 9 barrel segments. The extruder was provided with twin screws that incorporated kneading and mixing elements in an upstream melting zone and a downstream melt blending zone. All the polymer pellets and additive powders were pre-blended and fed at the main feed port of the extruder at a rate of nominally 250 g/min. The plasticizer Uniplex 214 was injected at barrel 6. Barrels were heated to a desired temperature profile of 200° C. at the feed port to a temperature ranging from 240-250° C. at the front end. The screw rpm was generally 300. The melt was extruded through a two hole die and was pelletized into granules.

The compounded formulations were pre-dried at 65° C. overnight in a dehumidifying dryer to provide a moisture level of less than 0.05% that is suitable for molding. The compounded formulations were molded into test pieces per ASTM D 638 for specification using a 180 ton Nissei Injection molding machine. The mold cavity included ASTM D638 type IV 3.2 mm thick tensile bars and type V 3.2 mm thick tensile bars. The barrel temperature profile was 220° C. at the feed port to 240° C. at the nozzle. Mold temperature was 70° C. Molded bars were ejected from the cavity and stored in dry-as-molded condition in vacuum sealed aluminum foiled bags until ready for testing.

### Tensile Test Methods Used in Table 6:

Tensile properties at 23° C. were measured per ASTM D638 specification using an Instron tensile tester model 4469. Crosshead speed was 50 mm/min. Tensile modulus and yield stress were recorded. Tensile properties at 125° C. were measured using a heating oven installed on the test machine with grips located inside the oven. Shorter ASTM D638 type V bars were used to accommodate higher elongation inside the oven. Crosshead speed was 250 mm/min. Tensile modulus at 125° C. was recorded.

DMA, DSC and salt resistance measurements were carried by the methods as described earlier for data in Table 3.

### Tube Extrusion & Burst Pressure Testing

Impact modified melt blended compositions were dried overnight in a dehumidifying dryer at 65 C. They were extruded into tubes measuring 8.3 mm OD X 6.3 mm ID using a Davis Standard tube extrusion system. The system consisted of a 50 mm single screw extruder equipped with a tubing die, a vacuum sizing tank with a plate style calibrator, puller and cutter. Die with bushing of 15.2 mm (0.600 in) and a tip of 8.9 mm (0.350 in) was used. Calibrator was 8.3 mm (0.327 in). Extruder barrel temperature profile was 210° C. at the feedport increasing to 220 to 230° C. at the die. Line speed was typically 4.6 m/min (15 ft/min). After establishing a stable process, tubing was cut to 30 cm long pieces and used for burst pressure measurements.

Burst pressure of the tubes was measured using a manual hydraulic pump fitted with a pressure gauge. One end of the tube was attached to the pump using a Swagelok fitting, while the other end of the tube was capped off. The burst pressure was measured by manually raising the fluid pressure till failure. Burst pressure at 125° C. was measured similarly by positioning the tube in a heated air circulating oven and allowing it to equilibrate to temperature for several hours prior to testing.

TABLE 6
Properties of Flexible Tube Composons

| Example | C11 | C12 | 19 | 20 |
|---|---|---|---|---|
| PA 610 | 66.5 | | | |
| PA612 (wt %) | | 66.5 | | |
| PA612/614 70/30 (wt %) | | | 66.5 | |
| PA614/616 70/30 (wt %) | | | | 66.5 |
| Fusabond N598 (wt %) | 23 | 23 | 23 | 23 |
| Uniplex 214 (wt %) | 7 | 7 | 7 | 7 |
| Carbon black | 2 | 2 | 2 | 2 |
| Naugard 445 | 0.5 | 0.5 | 0.5 | 0.5 |
| Lowinox 44B25 | 0.5 | 0.5 | 0.5 | 0.5 |
| IRGAFOS 168 | 0.5 | 0.5 | 0.5 | 0.5 |
| Tan Delta Peak Temp (° C.) | 45.3 | 44.3 | 24.9 | 25.4 |
| Tan Delta Peak Value | 0.13 | 0.11 | 0.12 | 0.12 |
| Storage modulus @ 23° C. (MPa) | 715 | 796 | 634 | 518 |
| Storage modulus @ 125° C. (MPa) | 116 | 149 | 125 | 106 |
| Tensile modulus @ 23° C. (MPa) | 646 | 726 | 591 | 507 |
| Tensile modulus @ 125° C. (Mpa) | 120 | 141 | 107 | 99 |
| Yield stress @ 23° C. max load (Mpa) | 46 | 37 | 32 | 31 |
| Burst Pressure @ 23° C. (bars) | 106 | 108 | 87 | 78 |
| 8.3 mm OD X 6.3 mm ID tubes | | | | |
| Burst Pressure @ 125° C. (bars) | 30 | 27 | 18 | 17 |
| 8.3 mm OD X 6.3 mm ID tubes | | | | |
| ZnCl2 resistance at 50 C. | 6 | 6-23 | 4 | 121 |
| (hours to first failure) | | | | |

We claim:

1. A copolyamide comprising at least two repeat units selected from the group consisting of structures (I) to (IV),
—C(O)(CH₂)₁₀C(O)NH(CH₂)ₙNH— (I);
—C(O)(CH₂)₁₂C(O)NH(CH₂)ₙNH— (II);
—C(O)(CH₂)₁₄C(O)NH(CH₂)ₙNH— (II);
and —C(O)(CH₂)₁₆C(O)NH(CH₂)ₙNH— (IV);
and n is an integer selected from 4, 6, 10 or 12; wherein at least one repeat unit is selected from structures (I) and (II); and any one the repeat units may be present up to about 92 mole percent.

2. The copolyamide of claim 1 consisting essentially of 4 to 92 mole percent of repeat units of structure (I), 4 to 92 mole percent of repeat units of structure (II), and 4 to 92 mole percent of repeat units of structure (III).

3. The copolyamide of claim 1 consisting essentially of 4 to 92 mole percent of repeat units of structure (I), 4 to 92 mole percent of repeat units of structure (II), and 4 to 92 mole percent of repeat units of structure (IV).

4. The copolyamide of claim 1 consisting essentially of 4 to 92 mole percent of repeat units of structure (I), 4 to 92 mole percent of repeat units of structure (III), and 4 to 92 mole percent of repeat units of structure (IV).

5. The copolyamide of claim 1 consisting essentially of 4 to 92 mole percent of repeat units of structure (II), 4 to 92 mole percent of repeat units of structure (III), and 4 to 92 mole percent of repeat units of structure (IV).

6. A process for making a copolyamide comprising at least two repeat units selected from the group consisting of structures (I) to (IV),
—C(O)(CH₂)₁₀C(O)NH(CH₂)ₙNH— (I);
—C(O)(CH₂)₁₂C(O)NH(CH₂)ₙNH— (II);
—C(O)(CH₂)₁₄C(O)NH(CH₂)ₙNH— (II);
and —C(O)(CH₂)₁₆C(O)NH(CH₂)ₙNH— (IV);
and n is an integer selected from 4, 6, 10 or 12; and any one the repeat units may be present up to about 92 mole percent, comprising the steps of;
a) fermenting a mixture of at least two long chain linear alkanes to a mixture of at least two long chain linear dicarboxylic acids of C12, C14, C16 and C18 carbon atoms;
b) polymerizing the mixture of at least two long chain dicarboxylic acids containing C12, C14, C16 and C18 carbon atoms and at least one diamine to provide said copolyamide.

7. The process of claim 6, wherein the long chain linear alkanes are derived by hydrotreating of vegetable oils selected from the group consisting of palm oil, palm kernel oil, rapeseed oil, soybean oil, cottonseed oil, peanut oil, olive oil, coconut oil, castor oil, canola oil, sunflower oil and a combination of vegetable oils.

8. The process of claim 6, wherein the long chain linear alkanes are derived by hydrotreating of fatty acids or esters of fatty acids derived from vegetable oils selected from the group consisting of palm oil, palm kernel oil, rapeseed oil, soybean oil, cottonseed oil, peanut oil, olive oil, coconut oil, castor oil, canola oil, sunflower oil and a combination of these.

9. The process of claim 6, wherein the long chain linear alkanes are derived by hydrotreating of animal fats selected from the group consisting of poultry fats, yellow grease, tallow, and a combination of these.

10. The process according to claim 6, wherein the fermentation of long chain linear alkanes is fermented with a transformed candila maltosa strain SW 81/82 identified as ATCC 74431.

11. The process according to claim 6, wherein the fermentation of long chain linear alkanes is fermented with a transformed candila maltosa strain SW 84/187.2 identified as ATCC 74430.

12. The process according to claim 6, wherein the fermentation of long chain linear alkanes is fermented with a transformed pichia pastoris strain SW 64/65 identified as ATCC 74409.

13. A thermoplastic composition comprising:
(a) a copolyamide comprising at least two repeat units selected from the group consisting of structures (I) to (iV),
—C(O)(CH₂)₁₀C(O)NH(CH₂)ₙNH— (I);
—C(O)(CH₂)₁₂C(O)NH(CH₂)ₙNH— (II);
—C(O)(CH₂)₁₄C(O)NH(CH₂)ₙNH— (II);
and —C(O)(CH₂)₁₆C(O)NH(CH₂)ₙNH— (IV);
and n is an integer selected from 4, 6, 10 or 12; wherein at least one repeat unit is selected from structures (I) and (II); and any one the repeat units may be present up to about 92 mole percent
(b) 0 to 30 weight percent of at least one polymeric toughener; and
(c) 0 to 10 weight percent of functional additives; and
(d) 0 to 60 weight percent of at least one reinforcing agent;
wherein the weight percent of (a), (b), (c) and (d) are based on the total weight of the thermoplastic composition and at least one component of the group consisting of (b), (c), (d), or a combination of these, is present in at least 0.1 weight percent.

14. A flexible tubing comprising:
(a) a copolyamide comprising at least two repeat units selected from the group consisting of structures (I) to (IV),
—C(O)(CH₂)₁₀C(O)NH(CH₂)ₙNH— (I);
—C(O)(CH₂)₁₂C(O)NH(CH₂)ₙNH— (II);
—C(O)(CH₂)₁₄C(O)NH(CH₂)ₙNH— (II);
and —C(O)(CH₂)₁₆C(O)NH(CH₂)ₙNH— (IV);
and n is an integer selected from 4, 6, 10 or 12; wherein at least one repeat unit is selected from structures (I) and (II); and any one the repeat units may be present up to about 92 mole percent;
(b) 0 to 30 weight percent of at least one polymeric toughener;
(d) 0 to 10 weight percent thermal stabilizer; and
(e) 0 to 20 weight percent of plasticizer; and
wherein the weight percent of (a), (b), and (d) and (e) are based on the total weight of the thermoplastic composition.
